(19) United States
(12) Patent Application Publication
(10) Pub. No.: US 2025/0359580 A1
(43) Pub. Date: Nov. 27, 2025

(54) DOUBLE-LAYER COATED MICROPARTICLES AND PREPARATION THEREOF

(71) Applicant: ADVANCED BIONUTRITION CORP., Columbia, MD (US)

(72) Inventors: Mordechai HAREL, Pikesville, MD (US); Majid KESHTMAND, Baltimore, MD (US); Trisha RICE, Columbia, MD (US)

(73) Assignee: ADVANCED BIONUTRITION CORP., Columbia, MD (US)

(21) Appl. No.: 18/873,595

(22) PCT Filed: May 18, 2023
(86) PCT No.: PCT/US2023/022700

§ 371 Date: Dec. 10, 2024

Related U.S. Application Data
(60) Provisional application No. 63/355,178, filed on Jun. 24, 2022.

(51) Int. Cl. A23P 10/35; A21D 2/14; A21D 8/04; A23L 29/00; A23L 31/10; A61K 9/50; A61K 31/07

(52) U.S. Cl. CPC A23P 10/35; A21D 2/14; A21D 8/042; A21D 8/047; A23L 29/06; A23L 31/10; A61K 9/5015; A61K 9/5073; A61K 31/07

(57) ABSTRACT

A double-layer coated microparticle for protecting a bioactive substance is provided. The double-layer coated microparticle comprises a core, an inner layer and an outer layer, wherein the core is coated with the inner layer and the outer layer, the inner layer is between the core and the outer layer, the core comprises the bioactive substance, the inner layer comprises a hydrophobic antioxidant, and the outer layer comprises a solid fat. Also provided is a method for preparing the double-layer coated microparticle. The preparation method comprises contacting the core with an inner layer solution to form the inner layer on the core, whereby an inner-layer coated microparticle is obtained; and contacting the inner-layer coated microparticle with an outer layer solution to form the outer layer on the inner-layer coated microparticle.

### CROSS-REFERENCE TO RELATED APPLICATION

This application claims priority to U.S. Provisional Application No. 63/355,178, filed Jun. 24, 2022, and the contents of which are incorporated herein by reference in their entireties for all purposes.

### FIELD OF THE INVENTION

The present invention relates to double-layer coated microparticles for protecting bioactive substances from heat, humidity, oxidation and acidic incursions.

### BACKGROUND OF THE INVENTION

A common difficulty associated with the incorporation of bioactive and functional substances and/or drugs in food products is loss of activity over time because, for example, decomposition during the food manufacturing process and/or inactivation during storage. The harsh environment of some food processes, like milling, mixing, baking, and extrusion, destroys many bioactive substances before they are incorporated into finished food products. Additional problems result from the interaction between desired bioactive substances and other food components, such as ingredients having a low or high pH, metal chelators, surfactants, and hygroscopic ingredients. This is especially true for living micro-organisms, enzymes and vitamins that are sensitive to most types of conventional food processing. Therefore, the food industry is continuously searching for new compositions and methods for protecting bioactive and functional substances against decomposition during processing and storage.

One method to protect and preserve the potency of a bioactive substance is encapsulation of the bioactive substance with a protective coating. Encapsulation is also used to protect a bioactive substance from oxygen, water, and light, as well as to convert the bioactive substance into a free-flowing powder that can be readily incorporated into various food products. Numerous attempts have been made over the years to enrobe or embed bioactive substances in many different types of biopolymers or synthetic polymers, including proteins, carbohydrates, and solid fats.

Typical methods of encapsulation include spray drying, air suspension coating, pan coating, spray cooling and chilling, agglomeration, and extrusion. However, these types of encapsulation are not suitable for protecting bioactive substances in food products that contain water at a low pH or have a high water activity because of oxidation and subsequent degradation of the encapsulated bioactive substances under aqueous conditions. Since water is involved in the preparation of most food products in at least one stage of the food manufacturing process and storage, encapsulation of bioactive substances in water-soluble polymers has limited applicability for improving protection of the bioactive compounds from humidity and free radicals, or for controlling retention of the bioactive substances and directing their release in a controlled manner.

Particles of a bioactive substance may be encapsulated with a single protective layer of solid fats to protect the bioactive substance in a humid environment during processing or storage. For example, particles containing a bioactive substance may be dispersed in a solid fat. For example, a carnauba wax coating may be applied onto polymeric shell encapsulating particles of a bioactive substance to improve shell strength and moisture resistance. A hydrophobic coating component may have a melting point (mp), also referred to as melting temperature, of about 70° C.

However, one major problem associated with these types of microencapsulation is that the coat is easily ruptured when water is added during a conventional food manufacturing process. Another problem associated with the use of solid fat encapsulation or coating is its limitation to food products that are processed at a temperature below the melting point of the solid fat. For example, solid fat coating is not applicable for a food process that includes boiling, baking, spray drying, or extruding, where the process temperature is well over 70° C. such that the coating fat becomes liquefied and its protective properties are lost. Moreover, since the melting point of solid fats to be used is very high (70° C. or higher), temperature sensitive bioactive substances such as living microorganisms and enzymes could be damaged, inactivated or destroyed during the coating process.

There remains a need for compositions and methods for coating a bioactive substance that overcome the problems associated with a single or multilayer coating comprised of sold fats.

### SUMMARY OF THE INVENTION

The present invention provides double-layer coated microparticles and preparation thereof.

A double-layer coated microparticle for protecting a bioactive substance is provided. The double-layer coated microparticle comprises a core, an inner layer and an outer layer. The core is coated with the inner layer and the outer layer. The inner layer is between the core and the outer layer. The core comprises the bioactive substance. The inner layer comprises a hydrophobic antioxidant. The outer layer comprises a solid fat.

The bioactive substance may be selected from the group consisting of probiotic bacteria, yeast, fungi, viruses, vaccines, nutraceuticals, pharmaceuticals, agro-biologicals, agro-chemicals, vitamins, enzymes, and food additives.

The double-layer coated microparticle may comprise the inner layer at 1-25 wt % of the double-layer coated microparticle.

The hydrophobic antioxidant may be selected from the group consisting of tocopherols, ascorbyl palmitate, tetrahexyldecyl ascorbate, vitamin A and derivatives thereof, carotenoids, quercetin and derivatives thereof, phenolic compounds, phyto-nutrients, herbs extracts and a combination thereof.

The inner layer may further comprises an emulsifier.

The double-layer coated microparticle may comprise the outer layer at 20-95 wt % of the double-layer coated microparticle.

The solid fat may be selected from the group consisting of animal fats, vegetable fats, natural waxes, hydrocarbon waxes, metallic stearates, solid polyethylene glycols (PEGs>1000) and a combination thereof.

The double-layer coated microparticle may have a particle size of 500-10,000 μm.

The double-layer coated microparticle may be stored at a temperature below the melting temperature of the solid fat.

A method for preparing a double-layer coated microparticle is also prepared. The preparation method comprises (a) contacting a core with an inner layer solution to form an inner layer on the core, wherein the core comprises a bioactive substance, and the inner layer comprises a hydrophobic antioxidant, whereby an inner-layer coated microparticle is obtained; and (b) contacting the inner-layer coated microparticle from step (a) with an outer layer solution to form an outer layer on the inner-layer coated microparticle, wherein the outer layer comprises a solid fat. As a result, a double-layer coated microparticle is prepared. Step (a) may be carried out at a temperature of 40-60° C. Step (b) may be carried out at a temperature above the melting temperature of the solid fat.

The preparation method may further comprise (c) storing the double-layer coated microparticle at a temperature below the melting temperature of the solid fat.

According to the preparation method, the bioactive substance is selected from the group consisting of probiotic bacteria, yeast, fungi, viruses, vaccines, nutraceuticals, pharmaceuticals, agro-biologicals, agro-chemicals, vitamins, enzymes, and food additives.

The preparation method may further comprise agglomerating, extruding or spray drying a mixture of the bioactive substance and an agglomerating agent so that the core is formed and has a particle size of 50-5,000 μm.

According to the preparation method, the double-layer coated microparticle may have a particle size of 500-10,000 μm. The double-layer coated microparticle may comprise the inner layer at 1-25 wt % of the coated microparticle. The hydrophobic antioxidant may be selected from the group consisting of tocopherols, ascorbyl palmitate, tetrahexyldecyl ascorbate, vitamin A and derivatives thereof, carotenoids, quercetin and derivatives thereof, phenolic compounds, phyto-nutrients, herbs extracts and a combination thereof. The inner layer may further comprise an emulsifier.

According to the preparation method, the coated microparticle may comprise the outer layer at 20-95 wt % of the coated microparticle. The solid fat may be selected from the group consisting of animal fats, vegetable fats, natural waxes, hydrocarbon waxes, metallic stearates, solid polyethylene glycols (PEGs>1000) and a combination thereof.

The preparation method may further comprise making a product with the double-layer coated microparticle. The product may be selected from the group consisting of pharmaceutical products, nutraceutical products, food products, feed products, and additive products for agriculture.

A double-layer coated microparticle prepared according to the preparation method of the present invention.

### BRIEF DESCRIPTION OF THE DRAWINGS

FIG. 1 shows microscopic views (5×) of coated microparticles. Cores containing vitamin A as a bioactive substance (left panel) were coated with an inner layer and then an outer layer to form double-layer coated microparticles (middle panel). The stability of the coated microparticles in canola oil was tested as evidenced by their microscopic views. Over 80% of the coated microparticles remained intact about 120 min after being mixed with the canola oil (right panel).

FIG. 2 shows microscopic views (5×) of bakery yeast (IDY) extruded cores (left panel) coated with a first (i.e., inner) layer (middle panel) and further with a second (i.e., outer) layer (right panel).

FIG. 3 shows proof time for yeast (IDY) cores (uncoated IDY), IDY cores coated with a single layer containing 100 wt % PEG-4000, which is a solid fat, (Sample #1), IDY cores coated with a single layer containing 2 wt % Vitamin E acetate 6 wt %, de-oiled soy lecithin, 20 wt % PEG-4000 25 wt % Palm kernel oil, 27 wt % stearic acid and 20 wt % magnesium stearate (Sample #2), and IDY cores coated with a double-layer, including an inner layer containing 11 wt % canola oil, 22 wt % Vitamin E acetate and 67 wt % de-oiled soy lecithin, and an outer layer containing 89 wt % PEG-4000 and 11 wt % magnesium stearate (Sample #3). The gassing power of the uncoated IDY and Samples #1-3 was measured after storing at 25° C. and 53% relative humidity (RH). After 0 or day 28, any one of the uncoated IDY or Samples #1-3 was incorporated into dough and proof time measured (i.e., time in minutes to reach 4× volume increase of a dough on).

FIG. 4 shows proof time for uncoated IDY cores and coated IDY microparticles with an inner layer and an outer layer, which were dispersed in an acidic medium and then stored in a refrigerator at about 4° C. The gassing power of the uncoated and coated IDY was measured over time and presented in proof time, i.e., time in minutes to reach 4× volume increase of a dough on day 9, 7, 14, 21 or 28 days.

### DETAILED DESCRIPTION OF THE INVENTION

The present invention provides microparticles having a double-layer coating for protecting a bioactive substance from undesirable conditions, for example, heat, oxidation, humidity, high water activity and an acidic environment. The double-layer coating includes an inner layer containing a hydrophobic antioxidant and an outer layer containing a solid fat. The invention is based on the inventors' surprising discovery of retention of integrity in oil for the double-layer coated microparticles containing a bioactive substance (e.g., vitamin A), and retention of the activity of a bioactive substance (e.g., an instant dry bakery yeast (IDY), savinase, vitamin A and microbe Trichoderma atroviride) in the double-layer coated microparticles upon exposure to an acidic medium or heat, or after storage. Also provided are methods for preparing and using the double-layer coated microparticles. Unless stated otherwise, all wt % figures herein are relative to the total composition.

The terms “encapsulate,” “coat,” and “enrobe” are used herein interchangeably and refer to covering the entire surface of a particle with one layer (single-layer), two layers (double-layer) or more than two layers (multi-layer) of coating. Each layer is formed by a composition comprising one or more substances. Two adjacent layers are formed by different compositions.

The term “particle size” as used herein refers to a diameter of a particle.

The term “water activity (Aw)” as used herein refers to the availability of water in a substance, for example, a composition of the present invention, which represents the energy status of water in the substance. It may be defined as the vapor pressure of water above a substance divided by that of pure water at the same temperature. Pure distilled water has a water activity of exactly one, i.e., Aw=1.0. A dry substance may have an Aw below about 0.5, 0.4, 0.3, 0.2 or 0.1.

The term “core” as used herein refers to a particle that is formed by a composition comprising a bioactive substance and is not encapsulated or coated. The core may have a particle size of about 50-5,000 μm, 50-1,000 μm, 50-500 μm, 100-5,000 μm, 100-1,000 μm, 100-500 μm, 200-5,000 μm, 200-1,000 μm, 200-500 μm, 500-5,000 μm, or 500-1,000 μm. The core may have a water activity (Aw) below about 0.5, 0.4, 0.3, 0.2 or 0.1.

The term “microparticle” as used herein refers to a particle having a particle size of about 50-10,000 μm, 50-5,000 μm, 50-1,000 μm, 50-500 μm, 100-10,000 μm, 100-5,000 μm, 100-1,000 μm, 100-500 μm, 200-10,000 μm, 200-5,000 μm, 200-1,000 μm, 200-500 μm, 500-10,000 μm, 500-5,000 μm, or 500-1,000 μm. The microparticles may be in the form of granules, beads, strands or any other solid accumulation. Granules may be agglomeration of microparticles. Beads may be round or cylindrical shaped microparticles. Strings may be elongated thin threads of microparticles. The microparticle may have a water activity (Aw) below 0.5, 0.4, 0.3, 0.2 or 0.1. The microparticle may be in a product, for example, food product. The core may constitute about 1-99, 1-92, 1-90, 1-85, 1-80, 1-75, 1-70, 1-65, 1-60, 1-55, 1-50, 1-45, 1-40, 1-35, 1-30, 1-25, 1-20, 1-15, 1-10, 1-5, 5-99, 5-95, 5-90, 5-85, 5-80, 5-75, 5-70, 5-65, 5-60, 5-55, 5-50, 5-45, 5-40, 5-35, 5-30, 5-25, 5-20, 5-15, 5-10, 10-99, 10-95, 10-90, 10-85, 10-80, 10-75, 10-70, 10-65, 10-60, 10-55, 10-50, 10-45, 10-40, 10-35, 10-30, 10-25, 10-20, 10-15, 15-99, 15-95, 15-90, 15-85, 15-80, 15-75, 15-70, 15-65, 15-60, 15-55, 15-50, 15-45, 15-40, 15-35, 15-30, 15-25, 15-20, 20-99, 20-95, 20-90, 20-85, 20-80, 20-75, 20-70, 20-65, 20-60, 20-55, 20-50, 20-45, 20-40, 20-35, 20-30, 20-25, 25-99, 25-95, 25-90, 25-85, 25-80, 25-75, 25-70, 25-65, 25-60, 25-55, 25-50, 25-45, 25-40, 25-35, 25-30, 30-99, 30-95, 30-90, 30-85, 30-80, 30-75, 30-70, 30-65, 30-60, 30-55, 30-50, 30-45, 30-40 or 30-35 wt %, at least about 1, 2, 3, 4, 5, 6, 7, 8, 9, 10, 15, 20, 25, 30, 35, 40, 45, 50, 55, 60, 65, 70, 75, 80, 85, 90, 95 or 99 wt %, or no more than about 1, 5, 10, 15, 20, 25, 30, 35, 40, 45, 50, 55, 60, 65, 70, 75, 80, 85, 90, 95 or 99 wt %, based on the total weight of the microparticle.

The term “coated microparticle” as used herein refers to a microparticle further comprising one or more layers of coating, and the core is encapsulated with the one or more layers of coating. The one or more layers of coating may have one layer (single-layer), two layers (double-layer) or more than two layers (multi-layer) encapsulating the core. Each layer is formed by a composition comprising one or more substances. Two adjacent layers are formed by different compositions. A microparticle comprising a double-layer of coating is also called double-layer coated microparticle. The layer(s) of coating may constitute about 1-99, 1-92, 1-90, 1-85, 1-80, 1-75, 1-70, 1-65, 1-60, 1-55, 1-50, 1-45, 1-40, 1-35, 1-30, 1-25, 1-20, 1-15, 1-10, 1-5, 5-99, 5-95, 5-90, 5-85, 5-80, 5-75, 5-70, 5-65, 5-60, 5-55, 5-50, 5-45, 5-40, 5-35, 5-30, 5-25, 5-20, 5-15, 5-10, 10-99, 10-95, 10-90, 10-85, 10-80, 10-75, 10-70, 10-65, 10-60, 10-55, 10-50, 10-45, 10-40, 10-35, 10-30, 10-25, 10-20, 10-15, 15-99, 15-95, 15-90, 15-85, 15-80, 15-75, 15-70, 15-65, 15-60, 15-55, 15-50, 15-45, 15-40, 15-35, 15-30, 15-25, 15-20, 20-99, 20-95, 20-90, 20-85, 20-80, 20-75, 20-70, 20-65, 20-60, 20-55, 20-50, 20-45, 20-40, 20-35, 20-30, 20-25, 25-99, 25-95, 25-90, 25-85, 25-80, 25-75, 25-70, 25-65, 25-60, 25-55, 25-50, 25-45, 25-40, 25-35, 25-30, 30-99, 30-95, 30-90, 30-85, 30-80, 30-75, 30-70, 30-65, 30-60, 30-55, 30-50, 30-45, 30-40 or 30-35 wt %, at least about 1, 2, 3, 4, 5, 6, 7, 8, 9, 10, 15, 20, 25, 30, 35, 40, 45, 50, 55, 60, 65, 70, 75, 80, 85, 90, 95 or 99 wt %, or no more than about 1, 5, 10, 15, 20, 25, 30, 35, 40, 45, 50, 55, 60, 65, 70, 75, 80, 85, 90, 95 or 99 wt %, based on the total weight of the coated microparticle.

The term “bioactive substance” as used refer to a compound, biological molecule, cell, tissue, organism, or a combination thereof. The cell, tissue or organism may be alive or dead. The bioactive substance may be selected from the group consisting of drugs, pharmaceuticals, nutraceuticals, chemicals, food, soil acidifier and combinations thereof. Examples of the bioactive substances include probiotic microbes, yeast, fungi, viruses, proteins or peptides (e.g., enzymes, vaccines, antibodies, and antimicrobial peptides), antibiotics, pesticides, herbicides, germicides, biocides, algicides, rodenticides, fungicides, insecticides, antioxidants, plant or animal growth promoters, plant or animal growth inhibitors, bio-fertilizers, bio-pesticides, drugs, vitamins, nucleic acids (e.g., RNA, DNA, Peptide Nucleic Acid, vectors, plasmids, ribozymes, aptamers, dendrimers, and the like). The bioactive substances may be selected from the group consisting of preservatives, nutraceuticals, disinfectants, catalysts, chemical reactants, fermentation agents, food or animal feed supplements, nutrients, flavors, colors, dyes, cosmetics, and other agents that provide improved functionality in food, feeds, and pharmaceutical products. The bioactive substance may be soluble or have limited solubility.

The term “hydrophobic antioxidant” as used herein refers to an oil soluble compound that interrupts a free radical reaction chain. Examples of the hydrophobic antioxidants include carotenoids (e.g., astaxanthin, zeaxanthin and lutein), carotenes (e.g., α-carotene, β-carotene and lycopene) and derivatives thereof, lipoic acid and derivatives thereof (e.g., dihydrolipoic acid), chelators (e.g., α-hydroxy fatty acids, palmitic acid, lauric acid, and medium chain triglycerides), quercetin and derivative thereof (e.g., dihydro quercetin), carmine (e.g., carminic acid), tocopherols and derivatives (e.g., vitamin E, vitamin E acetate and tocotrienol), ascorbyl palmitate, tetrahexyldecyl ascorbate, vitamin A and derivatives (e.g., vitamin A palmitate), hydrophobic phenolic compounds, rosemary, sage, oregano, thyme, ginger, summer savory, black pepper, red pepper, clove, marjoram, basil, peppermint, spearmint, common balm, fennel, parsley, cinnamon, curcumin, cumin, nutmeg, garlic, and coriander. The hydrophobic antioxidants may be natural or synthetic.

The term “solid fat” as used herein refers to an oil or fat having a melting point of about 25-90, 25-85, 25-80, 25-75, 25-70, 25-65, 25-60, 25-55, 25-50, 25-45, 25-40, 25-35, 25-30, 30-90, 30-85, 30-80, 30-75, 30-70, 30-65, 30-60, 30-55, 30-50, 30-45, 30-40, 30-35, 35-90, 35-85, 35-80, 35-75, 35-70, 35-65, 35-60, 35-55, 35-50, 35-45, 35-40, 40-90, 40-85, 40-80, 40-75, 40-70, 40-65, 40-60, 40-55, 40-50, 40-45, 45-90, 45-85, 40-80, 40-75, 40-70, 40-65, 40-60, 40-55, 40-50, 40-45, 45-90, 45-85, 45-80, 45-75, 45-70, 45-65, 45-60, 45-55, 45-50, 50-90, 50-85, 50-80, 50-75, 50-70, 50-65, 50-60, 50-55, 55-90, 55-85, 55-80, 55-75, 55-70, 55-65, 55-60, 60-90, 60-85, 60-80, 60-75, 60-70, 60-65, 65-90, 65-85, 65-80, 65-75, 65-70, 70-90, 70-85, 70-80, 70-75, 75-90, 75-85, 75-80, 80-90, 80-85 or 85-90° C. The solid fat may be from plants, algae, fungi, yeast, bacteria, or animals. The solid fat may be a hydrocarbon, which may be a natural product or synthetic. The solid fat may be an animal solid fat selected from the group consisting of beef tallow (mp 35-38° C.), mutton tallow (mp 40-45° C.), butter, cholesterol esters, stearine (mp 49-75° C.) and stearic acid (mp 71° C.). The solid fat may be a vegetable solid fat selected from the group consisting of hydrogenated oil, coconut oil, coconut butter, cocoa butter, cocoa butter equivalents, and phytosterol esters. The solid fat may be a natural wax selected from the group consisting of carnauba wax (mp 78-81° C.), candelilla wax (mp 68° C.), beeswax (mp 60-63° C.), spermaceti-sperm oil (mp 42-49° C.), Japan wax, jojoba oil and hardened jojoba oil (mp 30-40° C.). Examples of hydrocarbons (unsaponifiable) include paraffin wax (mp 35-36 C), montan wax (mp 76-84° C.), Ceresine wax (mp 60-85° C.), solid polyethylene glycols (Carbowax, PEGs>1000), and metallic stearates (e.g., aluminum stearates, magnesium stearates, sodium stearates, zinc stearate, calcium stearate).

The term “emulsifier” as used herein refers to a substance that stabilizes an emulsion. The emulsifier may comprise a lipophilic component and a hydrophilic component. Examples of emulsifiers include phospholipids, lecithins, monoglycerides, diglycerides, phosphated monoglycerides, esters of monoglycerides, succinic acid esters of monoglycerides, diacetyl tartaric acid esters of monoglycerides, sucrose esters of fatty acids, propylene glycol esters of fatty acids, sorbitan esters of fatty acids, and polyglycerol esters of fatty acids.

The present invention provides a double-layer coated microparticle for protecting a bioactive substance. The double-layer coated microparticle comprises a core, an inner layer and an outer layer. The core is coated with the inner layer and the outer layer. The inner layer is between the core and the outer layer. The core comprises the bioactive substance. The inner layer comprises a hydrophobic antioxidant. The outer layer comprises a solid fat. The inner layer may cover the entire surface of the core. The outer layer may cover the entire inner layer.

The core may have a particle size of about 50-5,000 μm, 50-1,000 μm, 50-500 μm, 100-5,000 μm, 100-1,000 μm, 100-500 μm, 200-5,000 μm, 200-1,000 μm, 200-500 μm, 500-5,000 μm, or 500-1,000 μm.

The core may constitute about 1-99, 1-92, 1-90, 1-85, 1-80, 1-75, 1-70, 1-65, 1-60, 1-55, 1-50, 1-45, 1-40, 1-35, 1-30, 1-25, 1-20, 1-15, 1-10, 1-5, 5-99, 5-95, 5-90, 5-85, 5-80, 5-75, 5-70, 5-65, 5-60, 5-55, 5-50, 5-45, 5-40, 5-35, 5-30, 5-25, 5-20, 5-15, 5-10, 10-99, 10-95, 10-90, 10-85, 10-80, 10-75, 10-70, 10-65, 10-60, 10-55, 10-50, 10-45, 10-40, 10-35, 10-30, 10-25, 10-20, 10-15, 15-99, 15-95, 15-90, 15-85, 15-80, 15-75, 15-70, 15-65, 15-60, 15-55, 15-50, 15-45, 15-40, 15-35, 15-30, 15-25, 15-20, 20-99, 20-95, 20-90, 20-85, 20-80, 20-75, 20-70, 20-65, 20-60, 20-55, 20-50, 20-45, 20-40, 20-35, 20-30, 20-25, 25-99, 25-95, 25-90, 25-85, 25-80, 25-75, 25-70, 25-65, 25-60, 25-55, 25-50, 25-45, 25-40, 25-35, 25-30, 30-99, 30-95, 30-90, 30-85, 30-80, 30-75, 30-70, 30-65, 30-60, 30-55, 30-50, 30-45, 30-40 or 30-35 wt %, at least about 1, 2, 3, 4, 5, 6, 7, 8, 9, 10, 15, 20, 25, 30, 35, 40, 45, 50, 55, 60, 65, 70, 75, 80, 85, 90, 95 or 99 wt %, or no more than about 1, 5, 10, 15, 20, 25, 30, 35, 40, 45, 50, 55, 60, 65, 70, 75, 80, 85, 90, 95 or 99 wt %, based on the total weight of the microparticle.

The core may comprise the bioactive substance at about 1-99, 1-92, 1-90, 1-85, 1-80, 1-75, 1-70, 1-65, 1-60, 1-55, 1-50, 1-45, 1-40, 1-35, 1-30, 1-25, 1-20, 1-15, 1-10, 1-5, 2-99, 2-92, 2-90, 2-85, 2-80, 2-75, 2-70, 2-65, 2-60, 2-55, 2-50, 2-45, 2-40, 2-35, 2-30, 2-25, 2-20, 2-15, 2-10, 2-5, 3-99, 3-92, 3-90, 3-85, 3-80, 3-75, 3-70, 3-65, 3-60, 3-55, 3-50, 3-45, 3-40, 3-35, 3-30, 3-25, 3-20, 3-15, 3-10, 3-5, 4-99, 4-92, 4-90, 4-85, 4-80, 4-75, 4-70, 4-65, 4-60, 4-55, 4-50, 4-45, 4-40, 4-35, 4-30, 4-25, 4-20, 4-15, 4-10, 4-5, 5-99, 5-95, 5-90, 5-85, 5-80, 5-75, 5-70, 5-65, 5-60, 5-55, 5-50, 5-45, 5-40, 5-35, 5-30, 5-25, 5-20, 5-15, 5-10, 6-99, 6-92, 6-90, 6-85, 6-80, 6-75, 6-70, 6-65, 6-60, 6-55, 6-50, 6-45, 6-40, 6-35, 6-30, 6-25, 6-20, 6-15, 6-10, 10-99, 10-95, 10-90, 10-85, 10-80, 10-75, 10-70, 10-65, 10-60, 10-55, 10-50, 10-45, 10-40, 10-35, 10-30, 10-25, 10-20, 10-15, 15-99, 15-95, 15-90, 15-85, 15-80, 15-75, 15-70, 15-65, 15-60, 15-55, 15-50, 15-45, 15-40, 15-35, 15-30, 15-25, 15-20, 20-99, 20-95, 20-90, 20-85, 20-80, 20-75, 20-70, 20-65, 20-60, 20-55, 20-50, 20-45, 20-40, 20-35, 20-30, 20-25, 25-99, 25-95, 25-90, 25-85, 25-80, 25-75, 25-70, 25-65, 25-60, 25-55, 25-50, 25-45, 25-40, 25-35, 25-30, 30-99, 30-95, 30-90, 30-85, 30-80, 30-75, 30-70, 30-65, 30-60, 30-55, 30-50, 30-45, 30-40 or 30-35 wt %, at least about 1, 2, 3, 4, 5, 6, 7, 8, 9, 10, 15, 20, 25, 30, 35, 40, 45, 50, 55, 60, 65, 70, 75, 80, 85, 90, 95 or 99 wt %, or no more than about 1, 5, 10, 15, 20, 25, 30, 35, 40, 45, 50, 55, 60, 65, 70, 75, 80, 85, 90, 95 or 99 wt %, based on the total weight of the core.

The bioactive substance may be a compound, biological molecule, cell, tissue, organism, or a combination thereof. The cell, tissue or organism may be alive or dead. The bioactive substance may be selected from the group consisting of drugs, pharmaceuticals, nutraceuticals, chemicals, food, soil acidifier and combinations thereof. Examples of the bioactive substances include probiotic microbes, yeast, fungi, viruses, proteins or peptides (e.g., enzymes, vaccines, antibodies, and antimicrobial peptides), antibiotics, pesticides, herbicides, germicides, biocides, algicides, rodenticides, fungicides, insecticides, antioxidants, plant or animal growth promoters, plant or animal growth inhibitors, bio-fertilizers, bio-pesticides, drugs, vitamins, nucleic acids (e.g., RNA, DNA, Peptide Nucleic Acid, vectors, plasmids, ribozymes, aptamers, dendrimers, and the like). The bioactive substances may be selected from the group consisting of preservatives, nutraceuticals, disinfectants, catalysts, chemical reactants, fermentation agents, food or animal feed supplements, nutrients, flavors, colors, dyes, cosmetics, and other agents that provide improved functionality in food, feeds, and pharmaceutical products. The bioactive substance may be selected from the group consisting of probiotic bacteria, yeast, fungi, viruses, vaccines, nutraceuticals, pharmaceuticals, agro-biologicals, agro-chemicals, vitamins, enzymes, and food additives.

The microparticle coated with the inner layer, also referred to as an inner-layer coated microparticle, may have a particle size of about 50-10,000 μm, 50-5,000 μm, 50-1,000 μm, 50-500 μm, 100-10,000 μm, 100-5,000 μm, 100-1,000 μm, 100-500 μm, 200-10,000 μm, 200-5,000 μm, 200-1,000 μm, 200-500 μm, 500-10,000 μm, 500-5,000 μm, or 500-1,000 μm.

The inner layer may constitute about 1-99, 1-92, 1-90, 1-85, 1-80, 1-75, 1-70, 1-65, 1-60, 1-55, 1-50, 1-45, 1-40, 1-35, 1-30, 1-25, 1-20, 1-15, 1-10, 1-5, 5-99, 5-95, 5-90, 5-85, 5-80, 5-75, 5-70, 5-65, 5-60, 5-55, 5-50, 5-45, 5-40, 5-35, 5-30, 5-25, 5-20, 5-15, 5-10, 10-99, 10-95, 10-90, 10-85, 10-80, 10-75, 10-70, 10-65, 10-60, 10-55, 10-50, 10-45, 10-40, 10-35, 10-30, 10-25, 10-20, 10-15, 15-99, 15-95, 15-90, 15-85, 15-80, 15-75, 15-70, 15-65, 15-60, 15-55, 15-50, 15-45, 15-40, 15-35, 15-30, 15-25, 15-20, 20-99, 20-95, 20-90, 20-85, 20-80, 20-75, 20-70, 20-65, 20-60, 20-55, 20-50, 20-45, 20-40, 20-35, 20-30, 20-25, 25-99, 25-95, 25-90, 25-85, 25-80, 25-75, 25-70, 25-65, 25-60, 25-55, 25-50, 25-45, 25-40, 25-35, 25-30, 30-99, 30-95, 30-90, 30-85, 30-80, 30-75, 30-70, 30-65, 30-60, 30-55, 30-50, 30-45, 30-40 or 30-35 wt %, at least about 1, 2, 3, 4, 5, 6, 7, 8, 9, 10, 15, 20, 25, 30, 35, 40, 45, 50, 55, 60, 65, 70, 75, 80, 85, 90, 95 or 99 wt %, or no more than about 1, 5, 10, 15, 20, 25, 30, 35, 40, 45, 50, 55, 60, 65, 70, 75, 80, 85, 90, 95 or 99 wt %, based on the total weight of the coated microparticle.

The inner layer may constitute about 1-99, 1-92, 1-90, 1-85, 1-80, 1-75, 1-70, 1-65, 1-60, 1-55, 1-50, 1-45, 1-40, 1-35, 1-30, 1-25, 1-20, 1-15, 1-10, 1-5, 5-99, 5-95, 5-90, 5-85, 5-80, 5-75, 5-70, 5-65, 5-60, 5-55, 5-50, 5-45, 5-40, 5-35, 5-30, 5-25, 5-20, 5-15, 5-10, 10-99, 10-95, 10-90, 10-85, 10-80, 10-75, 10-70, 10-65, 10-60, 10-55, 10-50, 10-45, 10-40, 10-35, 10-30, 10-25, 10-20, 10-15, 15-99, 15-95, 15-90, 15-85, 15-80, 15-75, 15-70, 15-65, 15-60, 15-55, 15-50, 15-45, 15-40, 15-35, 15-30, 15-25, 15-20, 20-99, 20-95, 20-90, 20-85, 20-80, 20-75, 20-70, 20-65, 20-60, 20-55, 20-50, 20-45, 20-40, 20-35, 20-30, 20-25, 25-99, 25-95, 25-90, 25-85, 25-80, 25-75, 25-70, 25-65, 25-60, 25-55, 25-50, 25-45, 25-40, 25-35, 25-30, 30-99, 30-95, 30-90, 30-85, 30-80, 30-75, 30-70, 30-65, 30-60, 30-55, 30-50, 30-45, 30-40 or 30-35 wt %, at least about 1, 2, 3, 4, 5, 6, 7, 8, 9, 10, 15, 20, 25, 30, 35, 40, 45, 50, 55, 60, 65, 70, 75, 80, 85, 90, 95 or 99 wt %, or no more than about 1, 5, 10, 15, 20, 25, 30, 35, 40, 45, 50, 55, 60, 65, 70, 75, 80, 85, 90, 95 or 99 wt %, based on the total weight of the inner layer and the outer layer.

The inner layer may comprise the hydrophobic antioxidant at about 1-99, 1-92, 1-90, 1-85, 1-80, 1-75, 1-70, 1-65, 1-60, 1-55, 1-50, 1-45, 1-40, 1-35, 1-30, 1-25, 1-20, 1-15, 1-10, 1-5, 2-99, 2-92, 2-90, 2-85, 2-80, 2-75, 2-70, 2-65, 2-60, 2-55, 2-50, 2-45, 2-40, 2-35, 2-30, 2-25, 2-20, 2-15, 2-10, 2-5, 3-99, 3-92, 3-90, 3-85, 3-80, 3-75, 3-70, 3-65, 3-60, 3-55, 3-50, 3-45, 3-40, 3-35, 3-30, 3-25, 3-20, 3-15, 3-10, 3-5, 4-99, 4-92, 4-90, 4-85, 4-80, 4-75, 4-70, 4-65, 4-60, 4-55, 4-50, 4-45, 4-40, 4-35, 4-30, 4-25, 4-20, 4-15, 4-10, 4-5, 5-99, 5-95, 5-90, 5-85, 5-80, 5-75, 5-70, 5-65, 5-60, 5-55, 5-50, 5-45, 5-40, 5-35, 5-30, 5-25, 5-20, 5-15, 5-10, 6-99, 6-92, 6-90, 6-85, 6-80, 6-75, 6-70, 6-65, 6-60, 6-55, 6-50, 6-45, 6-40, 6-35, 6-30, 6-25, 6-20, 6-15, 6-10, 10-99, 10-95, 10-90, 10-85, 10-80, 10-75, 10-70, 10-65, 10-60, 10-55, 10-50, 10-45, 10-40, 10-35, 10-30, 10-25, 10-20, 10-15, 15-99, 15-95, 15-90, 15-85, 15-80, 15-75, 15-70, 15-65, 15-60, 15-55, 15-50, 15-45, 15-40, 15-35, 15-30, 15-25, 15-20, 20-99, 20-95, 20-90, 20-85, 20-80, 20-75, 20-70, 20-65, 20-60, 20-55, 20-50, 20-45, 20-40, 20-35, 20-30, 20-25, 25-99, 25-95, 25-90, 25-85, 25-80, 25-75, 25-70, 25-65, 25-60, 25-55, 25-50, 25-45, 25-40, 25-35, 25-30, 30-99, 30-95, 30-90, 30-85, 30-80, 30-75, 30-70, 30-65, 30-60, 30-55, 30-50, 30-45, 30-40 or 30-35 wt %, at least about 1, 2, 3, 4, 5, 6, 7, 8, 9, 10, 15, 20, 25, 30, 35, 40, 45, 50, 55, 60, 65, 70, 75, 80, 85, 90, 95 or 99 wt %, or no more than about 1, 5, 10, 15, 20, 25, 30, 35, 40, 45, 50, 55, 60, 65, 70, 75, 80, 85, 90, 95 or 99 wt %, based on the total weight of the inner layer.

The hydrophobic antioxidant may be selected from the group consisting of carotenoids (e.g., astaxanthin, zeaxanthin and lutein), carotenes (e.g., α-carotene, β-carotene and lycopene) and derivatives thereof, lipoic acid and derivatives thereof (e.g., dihydrolipoic acid), chelators (e.g., α-hydroxy fatty acids, palmitic acid, lauric acid, and medium chain triglycerides), quercetin and derivative thereof (e.g., dihydro quercetin), carmine (e.g., carminic acid), tocopherols and derivatives (e.g., vitamin E, vitamin E acetate and tocotrienol), ascorbyl palmitate, tetrahexyldecyl ascorbate, vitamin A and derivatives (e.g., vitamin A palmitate), phenolic compounds, rosemary, sage, oregano, thyme, ginger, summer savory, black pepper, red pepper, clove, marjoram, basil, peppermint, spearmint, common balm, fennel, parsley, cinnamon, curcumin, cumin, nutmeg, garlic, coriander. The hydrophobic antioxidant may be selected from the group consisting of tocopherols, ascorbyl palmitate, tetrahexyldecyl ascorbate, vitamin A and derivatives thereof, carotenoids, quercetin and derivatives thereof, phenolic compounds, phyto-nutrients, herbs extracts and a combination thereof. The hydrophobic antioxidants may be natural or synthetic.

The inner layer may further comprise an emulsifier. The emulsifier may comprise a lipophilic component and a hydrophilic component. Examples of emulsifiers include phospholipids, lecithins, monoglycerides, diglycerides, phosphated monoglycerides, esters of monoglycerides, succinic acid esters of monoglycerides, diacetyl tartaric acid esters of monoglycerides, sucrose esters of fatty acids, propylene glycol esters of fatty acids, sorbitan esters of fatty acids, and polyglycerol esters of fatty acids. The emulsifier may be selected from the group consisting of mono and di-glycerides, phospholipids, egg lecithin, soy lecithin, sucrose fatty acid esters, polyglycerol fatty acid esters and a combination thereof.

The inner layer may comprise the emulsifier at about 1-99, 1-92, 1-90, 1-85, 1-80, 1-75, 1-70, 1-65, 1-60, 1-55, 1-50, 1-45, 1-40, 1-35, 1-30, 1-25, 1-20, 1-15, 1-10, 1-5, 2-99, 2-92, 2-90, 2-85, 2-80, 2-75, 2-70, 2-65, 2-60, 2-55, 2-50, 2-45, 2-40, 2-35, 2-30, 2-25, 2-20, 2-15, 2-10, 2-5, 3-99, 3-92, 3-90, 3-85, 3-80, 3-75, 3-70, 3-65, 3-60, 3-55, 3-50, 3-45, 3-40, 3-35, 3-30, 3-25, 3-20, 3-15, 3-10, 3-5, 4-99, 4-92, 4-90, 4-85, 4-80, 4-75, 4-70, 4-65, 4-60, 4-55, 4-50, 4-45, 4-40, 4-35, 4-30, 4-25, 4-20, 4-15, 4-10, 4-5, 5-99, 5-95, 5-90, 5-85, 5-80, 5-75, 5-70, 5-65, 5-60, 5-55, 5-50, 5-45, 5-40, 5-35, 5-30, 5-25, 5-20, 5-15, 5-10, 6-99, 6-92, 6-90, 6-85, 6-80, 6-75, 6-70, 6-65, 6-60, 6-55, 6-50, 6-45, 6-40, 6-35, 6-30, 6-25, 6-20, 6-15, 6-10, 10-99, 10-95, 10-90, 10-85, 10-80, 10-75, 10-70, 10-65, 10-60, 10-55, 10-50, 10-45, 10-40, 10-35, 10-30, 10-25, 10-20, 10-15, 15-99, 15-95, 15-90, 15-85, 15-80, 15-75, 15-70, 15-65, 15-60, 15-55, 15-50, 15-45, 15-40, 15-35, 15-30, 15-25, 15-20, 20-99, 20-95, 20-90, 20-85, 20-80, 20-75, 20-70, 20-65, 20-60, 20-55, 20-50, 20-45, 20-40, 20-35, 20-30, 20-25, 25-99, 25-95, 25-90, 25-85, 25-80, 25-75, 25-70, 25-65, 25-60, 25-55, 25-50, 25-45, 25-40, 25-35, 25-30, 30-99, 30-95, 30-90, 30-85, 30-80, 30-75, 30-70, 30-65, 30-60, 30-55, 30-50, 30-45, 30-40 or 30-35 wt %, at least about 1, 2, 3, 4, 5, 6, 7, 8, 9, 10, 15, 20, 25, 30, 35, 40, 45, 50, 55, 60, 65, 70, 75, 80, 85, 90, 95 or 99 wt %, or no more than about 1, 5, 10, 15, 20, 25, 30, 35, 40, 45, 50, 55, 60, 65, 70, 75, 80, 85, 90, 95 or 99 wt %, based on the total weight of the inner layer.

The microparticle coated with the inner and outer layers, i.e., the double-layer coated microparticles, may have a particle size of about 50-10,000 μm, 50-5,000 μm, 50-1,000 μm, 50-500 μm, 100-10,000 μm, 100-5,000 μm, 100-1,000 μm, 100-500 μm, 200-10,000 μm, 200-5,000 μm, 200-1,000 μm, 200-500 μm, 500-10,000 μm, 500-5,000 μm, or 500-1,000 μm.

The outer layer may constitute about 1-99, 1-92, 1-90, 1-85, 1-80, 1-75, 1-70, 1-65, 1-60, 1-55, 1-50, 1-45, 1-40, 1-35, 1-30, 1-25, 1-20, 1-15, 1-10, 1-5, 5-99, 5-95, 5-90, 5-85, 5-80, 5-75, 5-70, 5-65, 5-60, 5-55, 5-50, 5-45, 5-40, 5-35, 5-30, 5-25, 5-20, 5-15, 5-10, 10-99, 10-95, 10-90, 10-85, 10-80, 10-75, 10-70, 10-65, 10-60, 10-55, 10-50, 10-45, 10-40, 10-35, 10-30, 10-25, 10-20, 10-15, 15-99, 15-95, 15-90, 15-85, 15-80, 15-75, 15-70, 15-65, 15-60, 15-55, 15-50, 15-45, 15-40, 15-35, 15-30, 15-25, 15-20, 20-99, 20-95, 20-90, 20-85, 20-80, 20-75, 20-70, 20-65, 20-60, 20-55, 20-50, 20-45, 20-40, 20-35, 20-30, 20-25, 25-99, 25-95, 25-90, 25-85, 25-80, 25-75, 25-70, 25-65, 25-60, 25-55, 25-50, 25-45, 25-40, 25-35, 25-30, 30-99, 30-95, 30-90, 30-85, 30-80, 30-75, 30-70, 30-65, 30-60, 30-55, 30-50, 30-45, 30-40 or 30-35 wt %, at least about 1, 2, 3, 4, 5, 6, 7, 8, 9, 10, 15, 20, 25, 30, 35, 40, 45, 50, 55, 60, 65, 70, 75, 80, 85, 90, 95 or 99 wt %, or no more than about 1, 5, 10, 15, 20, 25, 30, 35, 40, 45, 50, 55, 60, 65, 70, 75, 80, 85, 90, 95 or 99 wt %, based on the total weight of the coated microparticle.

The outer layer may constitute about 1-99, 1-92, 1-90, 1-85, 1-80, 1-75, 1-70, 1-65, 1-60, 1-55, 1-50, 1-45, 1-40, 1-35, 1-30, 1-25, 1-20, 1-15, 1-10, 1-5, 5-99, 5-95, 5-90, 5-85, 5-80, 5-75, 5-70, 5-65, 5-60, 5-55, 5-50, 5-45, 5-40, 5-35, 5-30, 5-25, 5-20, 5-15, 5-10, 10-99, 10-95, 10-90, 10-85, 10-80, 10-75, 10-70, 10-65, 10-60, 10-55, 10-50, 10-45, 10-40, 10-35, 10-30, 10-25, 10-20, 10-15, 15-99, 15-95, 15-90, 15-85, 15-80, 15-75, 15-70, 15-65, 15-60, 15-55, 15-50, 15-45, 15-40, 15-35, 15-30, 15-25, 15-20, 20-99, 20-95, 20-90, 20-85, 20-80, 20-75, 20-70, 20-65, 20-60, 20-55, 20-50, 20-45, 20-40, 20-35, 20-30, 20-25, 25-99, 25-95, 25-90, 25-85, 25-80, 25-75, 25-70, 25-65, 25-60, 25-55, 25-50, 25-45, 25-40, 25-35, 25-30, 30-99, 30-95, 30-90, 30-85, 30-80, 30-75, 30-70, 30-65, 30-60, 30-55, 30-50, 30-45, 30-40 or 30-35 wt %, at least about 1, 2, 3, 4, 5, 6, 7, 8, 9, 10, 15, 20, 25, 30, 35, 40, 45, 50, 55, 60, 65, 70, 75, 80, 85, 90, 95 or 99 wt %, or no more than about 1, 5, 10, 15, 20, 25, 30, 35, 40, 45, 50, 55, 60, 65, 70, 75, 80, 85, 90, 95 or 99 wt %, based on the total weight of the inner layer and the outer layer.

The outer layer may comprise the solid fat at about 1-99, 1-92, 1-90, 1-85, 1-80, 1-75, 1-70, 1-65, 1-60, 1-55, 1-50, 1-45, 1-40, 1-35, 1-30, 1-25, 1-20, 1-15, 1-10, 1-5, 2-99, 2-92, 2-90, 2-85, 2-80, 2-75, 2-70, 2-65, 2-60, 2-55, 2-50, 2-45, 2-40, 2-35, 2-30, 2-25, 2-20, 2-15, 2-10, 2-5, 3-99, 3-92, 3-90, 3-85, 3-80, 3-75, 3-70, 3-65, 3-60, 3-55, 3-50, 3-45, 3-40, 3-35, 3-30, 3-25, 3-20, 3-15, 3-10, 3-5, 4-99, 4-92, 4-90, 4-85, 4-80, 4-75, 4-70, 4-65, 4-60, 4-55, 4-50, 4-45, 4-40, 4-35, 4-30, 4-25, 4-20, 4-15, 4-10, 4-5, 5-99, 5-95, 5-90, 5-85, 5-80, 5-75, 5-70, 5-65, 5-60, 5-55, 5-50, 5-45, 5-40, 5-35, 5-30, 5-25, 5-20, 5-15, 5-10, 6-99, 6-92, 6-90, 6-85, 6-80, 6-75, 6-70, 6-65, 6-60, 6-55, 6-50, 6-45, 6-40, 6-35, 6-30, 6-25, 6-20, 6-15, 6-10, 10-99, 10-95, 10-90, 10-85, 10-80, 10-75, 10-70, 10-65, 10-60, 10-55, 10-50, 10-45, 10-40, 10-35, 10-30, 10-25, 10-20, 10-15, 15-99, 15-95, 15-90, 15-85, 15-80, 15-75, 15-70, 15-65, 15-60, 15-55, 15-50, 15-45, 15-40, 15-35, 15-30, 15-25, 15-20, 20-99, 20-95, 20-90, 20-85, 20-80, 20-75, 20-70, 20-65, 20-60, 20-55, 20-50, 20-45, 20-40, 20-35, 20-30, 20-25, 25-99, 25-95, 25-90, 25-85, 25-80, 25-75, 25-70, 25-65, 25-60, 25-55, 25-50, 25-45, 25-40, 25-35, 25-30, 30-99, 30-95, 30-90, 30-85, 30-80, 30-75, 30-70, 30-65, 30-60, 30-55, 30-50, 30-45, 30-40 or 30-35 wt %, at least about 1, 2, 3, 4, 5, 6, 7, 8, 9, 10, 15, 20, 25, 30, 35, 40, 45, 50, 55, 60, 65, 70, 75, 80, 85, 90, 95 or 99 wt %, or no more than about 1, 5, 10, 15, 20, 25, 30, 35, 40, 45, 50, 55, 60, 65, 70, 75, 80, 85, 90, 95 or 99 wt %, based on the total weight of the outer layer.

The solid fat may be have a melting point of about 25-90, 25-85, 25-80, 25-75, 25-70, 25-65, 25-60, 25-55, 25-50, 25-45, 25-40, 25-35, 25-30, 30-90, 30-85, 30-80, 30-75, 30-70, 30-65, 30-60, 30-55, 30-50, 30-45, 30-40, 30-35, 35-90, 35-85, 35-80, 35-75, 35-70, 35-65, 35-60, 35-55, 35-50, 35-45, 35-40, 40-90, 40-85, 40-80, 40-75, 40-70, 40-65, 40-60, 40-55, 40-50, 40-45, 45-90, 45-85, 40-80, 40-75, 40-70, 40-65, 40-60, 40-55, 40-50, 40-45, 45-90, 45-85, 45-80, 45-75, 45-70, 45-65, 45-60, 45-55, 45-50, 50-90, 50-85, 50-80, 50-75, 50-70, 50-65, 50-60, 50-55, 55-90, 55-85, 55-80, 55-75, 55-70, 55-65, 55-60, 60-90, 60-85, 60-80, 60-75, 60-70, 60-65, 65-90, 65-85, 65-80, 65-75, 65-70, 70-90, 70-85, 70-80, 70-75, 75-90, 75-85, 75-80, 80-90, 80-85 or 85-90° C. The solid fat may be from plants, algae, fungi, yeast, bacteria, or animals. The solid fat may be a hydrocarbon, which may be a natural product or synthetic. The solid fat may be an animal solid fat selected from the group consisting of beef tallow (mp 35-38° C.), mutton tallow (mp 40-45° C.), butter, cholesterol esters, stearine (mp 49-75° C.) and stearic acid (mp 71° C.). The solid fat may be a vegetable solid fat selected from the group consisting of hydrogenated oil, coconut oil, coconut butter, cocoa butter, cocoa butter equivalents, and phytosterol esters. The solid fat may be a natural wax selected from the group consisting of carnauba wax (mp 78-81° C.), candelilla wax (mp 68° C.), beeswax (mp 60-63° C.), spermaceti-sperm oil (mp 42-49° C.), Japan wax, jojoba oil and hardened jojoba oil (mp 30-40° C.). Examples of hydrocarbons (unsaponifiable) include paraffin wax (mp 35-36 C), montan wax (mp 76-84° C.), Ceresine wax (mp 60-85° C.), solid polyethylene glycols (Carbowax, PEGs>1000), and metallic stearates (e.g., aluminum stearates, magnesium stearates, sodium stearates, zinc stearate, calcium stearate). The solid fat is selected from the group consisting of animal fats, vegetable fats, natural waxes, hydrocarbon waxes, metallic stearates, solid polyethylene glycols (PEGs>1000) and a combination thereof. The solid fat may be edible. Examples of edible solid fats include coconut oil, coconut butter, cocoa butter, cocoa butter equivalents, palm oil kernel, stearic acid and stearic acid slats, phytosterol esters and combinations thereof.

In one embodiment, the double-layer coated microparticle comprises a core, an inner layer and an outer layer. The core is coated with the inner layer and the outer layer. The inner layer is between the core and the outer layer. The core comprises the bioactive substance at 4-65 wt % of the weight of the core, the inner layer comprises a hydrophobic antioxidant at 1-80 wt % of the weight of the inner layer, and the outer layer comprises a solid fat at 20-95 wt % of the weight of outer layer.

The outer layer may be in a solid form in the coated microparticles, which may be incorporated into a food product containing the coated microparticles, when being stored. The final melting point of the outer layer may be adjusted by mixing two or more solid fats having different melting points. The outer layer in the coated microparticle may be converted from a liquid form into solid form when the temperature is reduced to room temperature (e.g., 20-25° C.). The outer layer in the coated microparticle may remain in a solid form when the coated microparticles are stored or incorporated into a food product.

The coated microparticle may be stored at a temperature below the melting point of the solid fat. The coated microparticle may be stored at a temperature of about 0-90, 0-85, 0-80, 0-75, 0-70, 0-65, 0-60, 0-55, 0-50, 0-45, 0-40, 0-35, 0-30, 30-90, 30-85, 30-80, 30-75, 30-70, 30-65, 30-60, 30-55, 30-50, 30-45, 30-40, 30-35, 35-90, 35-85, 35-80, 35-75, 35-70, 35-65, 35-60, 35-55, 35-50, 35-45, 35-40, 40-90, 40-85, 40-80, 40-75, 40-70, 40-65, 40-60, 40-55, 40-50, 40-45, 45-90, 45-85, 40-80, 40-75, 40-70, 40-65, 40-60, 40-55, 40-50, 40-45, 45-90, 45-85, 45-80, 45-75, 45-70, 45-65, 45-60, 45-55, 45-50, 50-90, 50-85, 50-80, 50-75, 50-70, 50-65, 50-60, 50-55, 55-90, 55-85, 55-80, 55-75, 55-70, 55-65, 55-60, 60-90, 60-85, 60-80, 60-75, 60-70, 60-65, 65-90, 65-85, 65-80, 65-75, 65-70, 70-90, 70-85, 70-80, 70-75, 75-90, 75-85, 75-80, 80-90, 80-85 or 85-90° C., below about 20, 25, 30, 35, 40, 45, 50, 55, 60, 65, 70, 75, 80, 85 or 90° C., or above 1, 5, 10, 15, 20, 25, 30, 35, 40, 45, 50, 55, 60, 65 or 70° C.

In the double-layer coated microparticle, the inner layer may be in contact with the core and the outer layer may be in contact with the inner layer.

The double-layer coated microparticle may be dry. The double-layer coated microparticle may have a water activity (Aw) no more than 0.5, 0.3, 0.2 or 0.1.

The double-layer coated microparticle may be stored under predetermined storage conditions for a predetermined period of time. The composition may be stored at a temperature below the melting point of the solid fat.

The predetermined storage conditions may include a predetermined temperature and a predetermined relative humidity (RH). The term “relative humidity (RH)” as used herein refers to the amount of water vapor in the air, often at a given temperature. Relative humidity is usually less than that required to saturate the air and is often expressed in percentage of saturation humidity. The predetermined temperature may be at least about 0, 5, 10, 15, 20, 25, 37, 40, 45, 50 or 55° C. The predetermined relative humidity (RH) may be at least about 10%, 20%, 30%, 33%, 35%, 40%, 50%, 60%, 70% or 80%. The predetermined conditions may be accelerated storage conditions. For example, the predetermined conditions may include about 5° C. and about 80% RH, about 40° C. and about 33% RH, or about 45° C. and about 33% RH.

The predetermined period of time may be at least about 1, 2, 3 or 4 weeks, or 1, 2, 3, 4, 5, 6, 12, 18, 24 or 36 months, preferably at least about 1, 2 or 3 months, more preferably at least about 1 or 3 months. A specified time period may include a shorter or longer time period that is within 10% of the specified time period. The term “3 months” as used herein refers to a time period of about 84-90 days. The term “2 months” as used herein refers to a time period of about 56-60 days. The term “1 month” as used herein refers to a time period of about 28-30 days.

After storage, at least about 10, 15, 20, 25, 30, 35, 40, 45, 50, 55, 60, 65, 70, 75, 80, 85, 90, 95 or 99% of the double-layer coated microparticle remain intact.

After storage, the double-layer coated microparticle may retain about 1-99, 1-92, 1-90, 1-85, 1-80, 1-75, 1-70, 1-65, 1-60, 1-55, 1-50, 1-45, 1-40, 1-35, 1-30, 1-25, 1-20, 1-15, 1-10, 1-5, 5-99, 5-95, 5-90, 5-85, 5-80, 5-75, 5-70, 5-65, 5-60, 5-55, 5-50, 5-45, 5-40, 5-35, 5-30, 5-25, 5-20, 5-15, 5-10, 10-99, 10-95, 10-90, 10-85, 10-80, 10-75, 10-70, 10-65, 10-60, 10-55, 10-50, 10-45, 10-40, 10-35, 10-30, 10-25, 10-20, 10-15, 15-99, 15-95, 15-90, 15-85, 15-80, 15-75, 15-70, 15-65, 15-60, 15-55, 15-50, 15-45, 15-40, 15-35, 15-30, 15-25, 15-20, 20-99, 20-95, 20-90, 20-85, 20-80, 20-75, 20-70, 20-65, 20-60, 20-55, 20-50, 20-45, 20-40, 20-35, 20-30, 20-25, 25-99, 25-95, 25-90, 25-85, 25-80, 25-75, 25-70, 25-65, 25-60, 25-55, 25-50, 25-45, 25-40, 25-35, 25-30, 30-99, 30-95, 30-90, 30-85, 30-80, 30-75, 30-70, 30-65, 30-60, 30-55, 30-50, 30-45, 30-40 or 30-35 wt %, at least about 1, 2, 3, 4, 5, 6, 7, 8, 9, 10, 15, 20, 25, 30, 35, 40, 45, 50, 55, 60, 65, 70, 75, 80, 85, 90, 95 or 99 wt %, or no more than about 1, 5, 10, 15, 20, 25, 30, 35, 40, 45, 50, 55, 60, 65, 70, 75, 80, 85, 90, 95 or 99 wt % of the total weight of the double-layer coated microparticles.

After storage, the double-layer coated microparticle may retain about 1-99, 1-92, 1-90, 1-85, 1-80, 1-75, 1-70, 1-65, 1-60, 1-55, 1-50, 1-45, 1-40, 1-35, 1-30, 1-25, 1-20, 1-15, 1-10, 1-5, 5-99, 5-95, 5-90, 5-85, 5-80, 5-75, 5-70, 5-65, 5-60, 5-55, 5-50, 5-45, 5-40, 5-35, 5-30, 5-25, 5-20, 5-15, 5-10, 10-99, 10-95, 10-90, 10-85, 10-80, 10-75, 10-70, 10-65, 10-60, 10-55, 10-50, 10-45, 10-40, 10-35, 10-30, 10-25, 10-20, 10-15, 15-99, 15-95, 15-90, 15-85, 15-80, 15-75, 15-70, 15-65, 15-60, 15-55, 15-50, 15-45, 15-40, 15-35, 15-30, 15-25, 15-20, 20-99, 20-95, 20-90, 20-85, 20-80, 20-75, 20-70, 20-65, 20-60, 20-55, 20-50, 20-45, 20-40, 20-35, 20-30, 20-25, 25-99, 25-95, 25-90, 25-85, 25-80, 25-75, 25-70, 25-65, 25-60, 25-55, 25-50, 25-45, 25-40, 25-35, 25-30, 30-99, 30-95, 30-90, 30-85, 30-80, 30-75, 30-70, 30-65, 30-60, 30-55, 30-50, 30-45, 30-40 or 30-35 wt %, at least about 1, 2, 3, 4, 5, 6, 7, 8, 9, 10, 15, 20, 25, 30, 35, 40, 45, 50, 55, 60, 65, 70, 75, 80, 85, 90, 95 or 99 wt %, or no more than about 1, 5, 10, 15, 20, 25, 30, 35, 40, 45, 50, 55, 60, 65, 70, 75, 80, 85, 90, 95 or 99 wt % of the total weight of the bioactive substance.

After storage, the double-layer coated microparticle may retain about 1-99, 1-92, 1-90, 1-85, 1-80, 1-75, 1-70, 1-65, 1-60, 1-55, 1-50, 1-45, 1-40, 1-35, 1-30, 1-25, 1-20, 1-15, 1-10, 1-5, 5-99, 5-95, 5-90, 5-85, 5-80, 5-75, 5-70, 5-65, 5-60, 5-55, 5-50, 5-45, 5-40, 5-35, 5-30, 5-25, 5-20, 5-15, 5-10, 10-99, 10-95, 10-90, 10-85, 10-80, 10-75, 10-70, 10-65, 10-60, 10-55, 10-50, 10-45, 10-40, 10-35, 10-30, 10-25, 10-20, 10-15, 15-99, 15-95, 15-90, 15-85, 15-80, 15-75, 15-70, 15-65, 15-60, 15-55, 15-50, 15-45, 15-40, 15-35, 15-30, 15-25, 15-20, 20-99, 20-95, 20-90, 20-85, 20-80, 20-75, 20-70, 20-65, 20-60, 20-55, 20-50, 20-45, 20-40, 20-35, 20-30, 20-25, 25-99, 25-95, 25-90, 25-85, 25-80, 25-75, 25-70, 25-65, 25-60, 25-55, 25-50, 25-45, 25-40, 25-35, 25-30, 30-99, 30-95, 30-90, 30-85, 30-80, 30-75, 30-70, 30-65, 30-60, 30-55, 30-50, 30-45, 30-40 or 30-35%, at least about 1, 2, 3, 4, 5, 6, 7, 8, 9, 10, 15, 20, 25, 30, 35, 40, 45, 50, 55, 60, 65, 70, 75, 80, 85, 90, 95 or 99%, or no more than about 1, 5, 10, 15, 20, 25, 30, 35, 40, 45, 50, 55, 60, 65, 70, 75, 80, 85, 90, 95 or 99% of the activity of the bioactive substance.

The present invention also provides a method for preparing a double-layer coated microparticle. The preparation method comprises a first contacting step and a second contacting step. The first contacting step comprises contacting a core with an inner layer solution to form an inner layer on the core so that an inner-layer coated microparticle is obtained. The second contacting step comprises contacting the inner-layer coated microparticle with an outer layer solution to form an outer layer on the inner-layer coated microparticle. As a result, a double-layer coated microparticle is prepared. The core comprises a bioactive substance. The inner layer comprises a hydrophobic antioxidant. The outer layer comprises a solid fat.

According to the preparation method, each double-layer coated microparticle comprises a core, an inner layer and an outer layer. The core is coated with the inner layer and the outer layer. The inner layer is between the core and the outer layer. The core comprises the bioactive substance. The inner layer comprises a hydrophobic antioxidant. The outer layer comprises a solid fat. The inner layer may cover the entire surface of the core. The outer layer may cover the entire inner layer.

According to the preparation method, the core may have a particle size of about 50-5,000 μm, 50-1,000 μm, 50-500 μm, 100-5,000 μm, 100-1,000 μm, 100-500 μm, 200-5,000 μm, 200-1,000 μm, 200-500 μm, 500-10,000 μm, 500-5,000 μm, or 500-1,000 μm. The core may constitute about 1-99, 1-92, 1-90, 1-85, 1-80, 1-75, 1-70, 1-65, 1-60, 1-55, 1-50, 1-45, 1-40, 1-35, 1-30, 1-25, 1-20, 1-15, 1-10, 1-5, 5-99, 5-95, 5-90, 5-85, 5-80, 5-75, 5-70, 5-65, 5-60, 5-55, 5-50, 5-45, 5-40, 5-35, 5-30, 5-25, 5-20, 5-15, 5-10, 10-99, 10-95, 10-90, 10-85, 10-80, 10-75, 10-70, 10-65, 10-60, 10-55, 10-50, 10-45, 10-40, 10-35, 10-30, 10-25, 10-20, 10-15, 15-99, 15-95, 15-90, 15-85, 15-80, 15-75, 15-70, 15-65, 15-60, 15-55, 15-50, 15-45, 15-40, 15-35, 15-30, 15-25, 15-20, 20-99, 20-95, 20-90, 20-85, 20-80, 20-75, 20-70, 20-65, 20-60, 20-55, 20-50, 20-45, 20-40, 20-35, 20-30, 20-25, 25-99, 25-95, 25-90, 25-85, 25-80, 25-75, 25-70, 25-65, 25-60, 25-55, 25-50, 25-45, 25-40, 25-35, 25-30, 30-99, 30-95, 30-90, 30-85, 30-80, 30-75, 30-70, 30-65, 30-60, 30-55, 30-50, 30-45, 30-40 or 30-35 wt %, at least about 1, 2, 3, 4, 5, 6, 7, 8, 9, 10, 15, 20, 25, 30, 35, 40, 45, 50, 55, 60, 65, 70, 75, 80, 85, 90, 95 or 99 wt %, or no more than about 1, 5, 10, 15, 20, 25, 30, 35, 40, 45, 50, 55, 60, 65, 70, 75, 80, 85, 90, 95 or 99 wt %, based on the total weight of the microparticle.

According to the preparation method, the core may comprise the bioactive substance at about 1-99, 1-92, 1-90, 1-85, 1-80, 1-75, 1-70, 1-65, 1-60, 1-55, 1-50, 1-45, 1-40, 1-35, 1-30, 1-25, 1-20, 1-15, 1-10, 1-5, 2-99, 2-92, 2-90, 2-85, 2-80, 2-75, 2-70, 2-65, 2-60, 2-55, 2-50, 2-45, 2-40, 2-35, 2-30, 2-25, 2-20, 2-15, 2-10, 2-5, 3-99, 3-92, 3-90, 3-85, 3-80, 3-75, 3-70, 3-65, 3-60, 3-55, 3-50, 3-45, 3-40, 3-35, 3-30, 3-25, 3-20, 3-15, 3-10, 3-5, 4-99, 4-92, 4-90, 4-85, 4-80, 4-75, 4-70, 4-65, 4-60, 4-55, 4-50, 4-45, 4-40, 4-35, 4-30, 4-25, 4-20, 4-15, 4-10, 4-5, 5-99, 5-95, 5-90, 5-85, 5-80, 5-75, 5-70, 5-65, 5-60, 5-55, 5-50, 5-45, 5-40, 5-35, 5-30, 5-25, 5-20, 5-15, 5-10, 6-99, 6-92, 6-90, 6-85, 6-80, 6-75, 6-70, 6-65, 6-60, 6-55, 6-50, 6-45, 6-40, 6-35, 6-30, 6-25, 6-20, 6-15, 6-10, 10-99, 10-95, 10-90, 10-85, 10-80, 10-75, 10-70, 10-65, 10-60, 10-55, 10-50, 10-45, 10-40, 10-35, 10-30, 10-25, 10-20, 10-15, 15-99, 15-95, 15-90, 15-85, 15-80, 15-75, 15-70, 15-65, 15-60, 15-55, 15-50, 15-45, 15-40, 15-35, 15-30, 15-25, 15-20, 20-99, 20-95, 20-90, 20-85, 20-80, 20-75, 20-70, 20-65, 20-60, 20-55, 20-50, 20-45, 20-40, 20-35, 20-30, 20-25, 25-99, 25-95, 25-90, 25-85, 25-80, 25-75, 25-70, 25-65, 25-60, 25-55, 25-50, 25-45, 25-40, 25-35, 25-30, 30-99, 30-95, 30-90, 30-85, 30-80, 30-75, 30-70, 30-65, 30-60, 30-55, 30-50, 30-45, 30-40 or 30-35 wt %, at least about 1, 2, 3, 4, 5, 6, 7, 8, 9, 10, 15, 20, 25, 30, 35, 40, 45, 50, 55, 60, 65, 70, 75, 80, 85, 90, 95 or 99 wt %, or no more than about 1, 5, 10, 15, 20, 25, 30, 35, 40, 45, 50, 55, 60, 65, 70, 75, 80, 85, 90, 95 or 99 wt %, based on the total weight of the core. The bioactive substance may be a compound, biological molecule, cell, tissue, organism, or a combination thereof. The cell, tissue or organism may be alive or dead. The bioactive substance may be selected from the group consisting of drugs, pharmaceuticals, nutraceuticals, chemicals, food, soil acidifier and combinations thereof. Examples of the bioactive substances include probiotic microbes, yeast, fungi, viruses, proteins or peptides (e.g., enzymes, vaccines, antibodies, and antimicrobial peptides), antibiotics, pesticides, herbicides, germicides, biocides, algicides, rodenticides, fungicides, insecticides, antioxidants, plant or animal growth promoters, plant or animal growth inhibitors, bio-fertilizers, bio-pesticides, drugs, vitamins, nucleic acids (e.g., RNA, DNA, Peptide Nucleic Acid, vectors, plasmids, ribozymes, aptamers, dendrimers, and the like). The bioactive substances may be selected from the group consisting of preservatives, nutraceuticals, disinfectants, catalysts, chemical reactants, fermentation agents, food or animal feed supplements, nutrients, flavors, colors, dyes, cosmetics, and other agents that provide improved functionality in food, feeds, and pharmaceutical products. The bioactive substance may be selected from the group consisting of probiotic bacteria, yeast, fungi, viruses, vaccines, nutraceuticals, pharmaceuticals, agro-biologicals, agro-chemicals, vitamins, enzymes, and food additives.

According to the preparation method, the microparticles coated with the inner layer may have a particle size of about 50-10,000 μm, 50-5,000 μm, 50-1,000 μm, 50-500 μm, 100-10,000 μm, 100-5,000 μm, 100-1,000 μm, 100-500 μm, 200-10,000 μm, 200-5,000 μm, 200-1,000 μm, 200-500 μm, 500-10,000 μm, 500-5,000 μm, or 500-1,000 μm.

According to the preparation method, the inner layer may constitute about 1-99, 1-92, 1-90, 1-85, 1-80, 1-75, 1-70, 1-65, 1-60, 1-55, 1-50, 1-45, 1-40, 1-35, 1-30, 1-25, 1-20, 1-15, 1-10, 1-5, 5-99, 5-95, 5-90, 5-85, 5-80, 5-75, 5-70, 5-65, 5-60, 5-55, 5-50, 5-45, 5-40, 5-35, 5-30, 5-25, 5-20, 5-15, 5-10, 10-99, 10-95, 10-90, 10-85, 10-80, 10-75, 10-70, 10-65, 10-60, 10-55, 10-50, 10-45, 10-40, 10-35, 10-30, 10-25, 10-20, 10-15, 15-99, 15-95, 15-90, 15-85, 15-80, 15-75, 15-70, 15-65, 15-60, 15-55, 15-50, 15-45, 15-40, 15-35, 15-30, 15-25, 15-20, 20-99, 20-95, 20-90, 20-85, 20-80, 20-75, 20-70, 20-65, 20-60, 20-55, 20-50, 20-45, 20-40, 20-35, 20-30, 20-25, 25-99, 25-95, 25-90, 25-85, 25-80, 25-75, 25-70, 25-65, 25-60, 25-55, 25-50, 25-45, 25-40, 25-35, 25-30, 30-99, 30-95, 30-90, 30-85, 30-80, 30-75, 30-70, 30-65, 30-60, 30-55, 30-50, 30-45, 30-40 or 30-35 wt %, at least about 1, 2, 3, 4, 5, 6, 7, 8, 9, 10, 15, 20, 25, 30, 35, 40, 45, 50, 55, 60, 65, 70, 75, 80, 85, 90, 95 or 99 wt %, or no more than about 1, 5, 10, 15, 20, 25, 30, 35, 40, 45, 50, 55, 60, 65, 70, 75, 80, 85, 90, 95 or 99 wt %, based on the total weight of the coated microparticle.

According to the preparation method, the inner layer may constitute about 1-99, 1-92, 1-90, 1-85, 1-80, 1-75, 1-70, 1-65, 1-60, 1-55, 1-50, 1-45, 1-40, 1-35, 1-30, 1-25, 1-20, 1-15, 1-10, 1-5, 5-99, 5-95, 5-90, 5-85, 5-80, 5-75, 5-70, 5-65, 5-60, 5-55, 5-50, 5-45, 5-40, 5-35, 5-30, 5-25, 5-20, 5-15, 5-10, 10-99, 10-95, 10-90, 10-85, 10-80, 10-75, 10-70, 10-65, 10-60, 10-55, 10-50, 10-45, 10-40, 10-35, 10-30, 10-25, 10-20, 10-15, 15-99, 15-95, 15-90, 15-85, 15-80, 15-75, 15-70, 15-65, 15-60, 15-55, 15-50, 15-45, 15-40, 15-35, 15-30, 15-25, 15-20, 20-99, 20-95, 20-90, 20-85, 20-80, 20-75, 20-70, 20-65, 20-60, 20-55, 20-50, 20-45, 20-40, 20-35, 20-30, 20-25, 25-99, 25-95, 25-90, 25-85, 25-80, 25-75, 25-70, 25-65, 25-60, 25-55, 25-50, 25-45, 25-40, 25-35, 25-30, 30-99, 30-95, 30-90, 30-85, 30-80, 30-75, 30-70, 30-65, 30-60, 30-55, 30-50, 30-45, 30-40 or 30-35 wt %, at least about 1, 2, 3, 4, 5, 6, 7, 8, 9, 10, 15, 20, 25, 30, 35, 40, 45, 50, 55, 60, 65, 70, 75, 80, 85, 90, 95 or 99 wt %, or no more than about 1, 5, 10, 15, 20, 25, 30, 35, 40, 45, 50, 55, 60, 65, 70, 75, 80, 85, 90, 95 or 99 wt %, based on the total weight of the inner layer and the outer layer.

According to the preparation method, the inner layer may comprise the hydrophobic antioxidant at about 1-99, 1-92, 1-90, 1-85, 1-80, 1-75, 1-70, 1-65, 1-60, 1-55, 1-50, 1-45, 1-40, 1-35, 1-30, 1-25, 1-20, 1-15, 1-10, 1-5, 2-99, 2-92, 2-90, 2-85, 2-80, 2-75, 2-70, 2-65, 2-60, 2-55, 2-50, 2-45, 2-40, 2-35, 2-30, 2-25, 2-20, 2-15, 2-10, 2-5, 3-99, 3-92, 3-90, 3-85, 3-80, 3-75, 3-70, 3-65, 3-60, 3-55, 3-50, 3-45, 3-40, 3-35, 3-30, 3-25, 3-20, 3-15, 3-10, 3-5, 4-99, 4-92, 4-90, 4-85, 4-80, 4-75, 4-70, 4-65, 4-60, 4-55, 4-50, 4-45, 4-40, 4-35, 4-30, 4-25, 4-20, 4-15, 4-10, 4-5, 5-99, 5-95, 5-90, 5-85, 5-80, 5-75, 5-70, 5-65, 5-60, 5-55, 5-50, 5-45, 5-40, 5-35, 5-30, 5-25, 5-20, 5-15, 5-10, 6-99, 6-92, 6-90, 6-85, 6-80, 6-75, 6-70, 6-65, 6-60, 6-55, 6-50, 6-45, 6-40, 6-35, 6-30, 6-25, 6-20, 6-15, 6-10, 10-99, 10-95, 10-90, 10-85, 10-80, 10-75, 10-70, 10-65, 10-60, 10-55, 10-50, 10-45, 10-40, 10-35, 10-30, 10-25, 10-20, 10-15, 15-99, 15-95, 15-90, 15-85, 15-80, 15-75, 15-70, 15-65, 15-60, 15-55, 15-50, 15-45, 15-40, 15-35, 15-30, 15-25, 15-20, 20-99, 20-95, 20-90, 20-85, 20-80, 20-75, 20-70, 20-65, 20-60, 20-55, 20-50, 20-45, 20-40, 20-35, 20-30, 20-25, 25-99, 25-95, 25-90, 25-85, 25-80, 25-75, 25-70, 25-65, 25-60, 25-55, 25-50, 25-45, 25-40, 25-35, 25-30, 30-99, 30-95, 30-90, 30-85, 30-80, 30-75, 30-70, 30-65, 30-60, 30-55, 30-50, 30-45, 30-40 or 30-35 wt %, at least about 1, 2, 3, 4, 5, 6, 7, 8, 9, 10, 15, 20, 25, 30, 35, 40, 45, 50, 55, 60, 65, 70, 75, 80, 85, 90, 95 or 99 wt %, or no more than about 1, 5, 10, 15, 20, 25, 30, 35, 40, 45, 50, 55, 60, 65, 70, 75, 80, 85, 90, 95 or 99 wt %, based on the total weight of the inner layer.

According to the preparation method, the hydrophobic antioxidant may be selected from the group consisting of carotenoids (e.g., astaxanthin, zeaxanthin and lutein), carotenes (e.g., α-carotene, β-carotene and lycopene) and derivatives thereof, lipoic acid and derivatives thereof (e.g., dihydrolipoic acid), chelators (e.g., α-hydroxy fatty acids, palmitic acid, lauric acid, and medium chain triglycerides), quercetin and derivative thereof (e.g., dihydro quercetin), carmine (e.g., carminic acid), tocopherols and derivatives (e.g., vitamin E, vitamin E acetate and tocotrienol), ascorbyl palmitate, tetrahexyldecyl ascorbate, vitamin A and derivatives (e.g., vitamin A palmitate), phenolic compounds, rosemary, sage, oregano, thyme, ginger, summer savory, black pepper, red pepper, clove, marjoram, basil, peppermint, spearmint, common balm, fennel, parsley, cinnamon, curcumin, cumin, nutmeg, garlic, coriander. The hydrophobic antioxidant may be selected from the group consisting of tocopherols, ascorbyl palmitate, tetrahexyldecyl ascorbate, vitamin A and derivatives thereof, carotenoids, quercetin and derivatives thereof, phenolic compounds, phyto-nutrients, herbs extracts and a combination thereof. The hydrophobic antioxidants may be natural or synthetic.

According to the preparation method, the inner layer may further comprise an emulsifier. The emulsifier may comprise a lipophilic component and a hydrophilic component. Examples of emulsifiers include phospholipids, lecithins, monoglycerides, diglycerides, phosphated monoglycerides, esters of monoglycerides, succinic acid esters of monoglycerides, diacetyl tartaric acid esters of monoglycerides, sucrose esters of fatty acids, propylene glycol esters of fatty acids, sorbitan esters of fatty acids, and polyglycerol esters of fatty acids. The emulsifier may be selected from the group consisting of mono and di-glycerides, phospholipids, egg lecithin, soy lecithin, sucrose fatty acid esters, polyglycerol fatty acid esters and a combination thereof. The inner layer may comprise the emulsifier at about 1-99, 1-92, 1-90, 1-85, 1-80, 1-75, 1-70, 1-65, 1-60, 1-55, 1-50, 1-45, 1-40, 1-35, 1-30, 1-25, 1-20, 1-15, 1-10, 1-5, 2-99, 2-92, 2-90, 2-85, 2-80, 2-75, 2-70, 2-65, 2-60, 2-55, 2-50, 2-45, 2-40, 2-35, 2-30, 2-25, 2-20, 2-15, 2-10, 2-5, 3-99, 3-92, 3-90, 3-85, 3-80, 3-75, 3-70, 3-65, 3-60, 3-55, 3-50, 3-45, 3-40, 3-35, 3-30, 3-25, 3-20, 3-15, 3-10, 3-5, 4-99, 4-92, 4-90, 4-85, 4-80, 4-75, 4-70, 4-65, 4-60, 4-55, 4-50, 4-45, 4-40, 4-35, 4-30, 4-25, 4-20, 4-15, 4-10, 4-5, 5-99, 5-95, 5-90, 5-85, 5-80, 5-75, 5-70, 5-65, 5-60, 5-55, 5-50, 5-45, 5-40, 5-35, 5-30, 5-25, 5-20, 5-15, 5-10, 6-99, 6-92, 6-90, 6-85, 6-80, 6-75, 6-70, 6-65, 6-60, 6-55, 6-50, 6-45, 6-40, 6-35, 6-30, 6-25, 6-20, 6-15, 6-10, 10-99, 10-95, 10-90, 10-85, 10-80, 10-75, 10-70, 10-65, 10-60, 10-55, 10-50, 10-45, 10-40, 10-35, 10-30, 10-25, 10-20, 10-15, 15-99, 15-95, 15-90, 15-85, 15-80, 15-75, 15-70, 15-65, 15-60, 15-55, 15-50, 15-45, 15-40, 15-35, 15-30, 15-25, 15-20, 20-99, 20-95, 20-90, 20-85, 20-80, 20-75, 20-70, 20-65, 20-60, 20-55, 20-50, 20-45, 20-40, 20-35, 20-30, 20-25, 25-99, 25-95, 25-90, 25-85, 25-80, 25-75, 25-70, 25-65, 25-60, 25-55, 25-50, 25-45, 25-40, 25-35, 25-30, 30-99, 30-95, 30-90, 30-85, 30-80, 30-75, 30-70, 30-65, 30-60, 30-55, 30-50, 30-45, 30-40 or 30-35 wt %, at least about 1, 2, 3, 4, 5, 6, 7, 8, 9, 10, 15, 20, 25, 30, 35, 40, 45, 50, 55, 60, 65, 70, 75, 80, 85, 90, 95 or 99 wt %, or no more than about 1, 5, 10, 15, 20, 25, 30, 35, 40, 45, 50, 55, 60, 65, 70, 75, 80, 85, 90, 95 or 99 wt %, based on the total weight of the inner layer.

According to the preparation method, the double-layer coated microparticles may have a particle size of about 50-10,000 μm, 50-5,000 μm, 50-1,000 μm, 50-500 μm, 100-10,000 μm, 100-5,000 μm, 100-1,000 μm, 100-500 μm, 200-10,000 μm, 200-5,000 μm, 200-1,000 μm, 200-500 μm, 500-10,000 μm, 500-5,000 μm, or 500-1,000 μm.

According to the preparation method, the outer layer may constitute about 1-99, 1-92, 1-90, 1-85, 1-80, 1-75, 1-70, 1-65, 1-60, 1-55, 1-50, 1-45, 1-40, 1-35, 1-30, 1-25, 1-20, 1-15, 1-10, 1-5, 5-99, 5-95, 5-90, 5-85, 5-80, 5-75, 5-70, 5-65, 5-60, 5-55, 5-50, 5-45, 5-40, 5-35, 5-30, 5-25, 5-20, 5-15, 5-10, 10-99, 10-95, 10-90, 10-85, 10-80, 10-75, 10-70, 10-65, 10-60, 10-55, 10-50, 10-45, 10-40, 10-35, 10-30, 10-25, 10-20, 10-15, 15-99, 15-95, 15-90, 15-85, 15-80, 15-75, 15-70, 15-65, 15-60, 15-55, 15-50, 15-45, 15-40, 15-35, 15-30, 15-25, 15-20, 20-99, 20-95, 20-90, 20-85, 20-80, 20-75, 20-70, 20-65, 20-60, 20-55, 20-50, 20-45, 20-40, 20-35, 20-30, 20-25, 25-99, 25-95, 25-90, 25-85, 25-80, 25-75, 25-70, 25-65, 25-60, 25-55, 25-50, 25-45, 25-40, 25-35, 25-30, 30-99, 30-95, 30-90, 30-85, 30-80, 30-75, 30-70, 30-65, 30-60, 30-55, 30-50, 30-45, 30-40 or 30-35 wt %, at least about 1, 2, 3, 4, 5, 6, 7, 8, 9, 10, 15, 20, 25, 30, 35, 40, 45, 50, 55, 60, 65, 70, 75, 80, 85, 90, 95 or 99 wt %, or no more than about 1, 5, 10, 15, 20, 25, 30, 35, 40, 45, 50, 55, 60, 65, 70, 75, 80, 85, 90, 95 or 99 wt %, based on the total weight of the coated microparticle.

According to the preparation method, the outer layer may constitute about 1-99, 1-92, 1-90, 1-85, 1-80, 1-75, 1-70, 1-65, 1-60, 1-55, 1-50, 1-45, 1-40, 1-35, 1-30, 1-25, 1-20, 1-15, 1-10, 1-5, 5-99, 5-95, 5-90, 5-85, 5-80, 5-75, 5-70, 5-65, 5-60, 5-55, 5-50, 5-45, 5-40, 5-35, 5-30, 5-25, 5-20, 5-15, 5-10, 10-99, 10-95, 10-90, 10-85, 10-80, 10-75, 10-70, 10-65, 10-60, 10-55, 10-50, 10-45, 10-40, 10-35, 10-30, 10-25, 10-20, 10-15, 15-99, 15-95, 15-90, 15-85, 15-80, 15-75, 15-70, 15-65, 15-60, 15-55, 15-50, 15-45, 15-40, 15-35, 15-30, 15-25, 15-20, 20-99, 20-95, 20-90, 20-85, 20-80, 20-75, 20-70, 20-65, 20-60, 20-55, 20-50, 20-45, 20-40, 20-35, 20-30, 20-25, 25-99, 25-95, 25-90, 25-85, 25-80, 25-75, 25-70, 25-65, 25-60, 25-55, 25-50, 25-45, 25-40, 25-35, 25-30, 30-99, 30-95, 30-90, 30-85, 30-80, 30-75, 30-70, 30-65, 30-60, 30-55, 30-50, 30-45, 30-40 or 30-35 wt %, at least about 1, 2, 3, 4, 5, 6, 7, 8, 9, 10, 15, 20, 25, 30, 35, 40, 45, 50, 55, 60, 65, 70, 75, 80, 85, 90, 95 or 99 wt %, or no more than about 1, 5, 10, 15, 20, 25, 30, 35, 40, 45, 50, 55, 60, 65, 70, 75, 80, 85, 90, 95 or 99 wt %, based on the total weight of the inner layer and the outer layer.

According to the preparation method, the outer layer may comprise the solid fat at about 1-99, 1-92, 1-90, 1-85, 1-80, 1-75, 1-70, 1-65, 1-60, 1-55, 1-50, 1-45, 1-40, 1-35, 1-30, 1-25, 1-20, 1-15, 1-10, 1-5, 2-99, 2-92, 2-90, 2-85, 2-80, 2-75, 2-70, 2-65, 2-60, 2-55, 2-50, 2-45, 2-40, 2-35, 2-30, 2-25, 2-20, 2-15, 2-10, 2-5, 3-99, 3-92, 3-90, 3-85, 3-80, 3-75, 3-70, 3-65, 3-60, 3-55, 3-50, 3-45, 3-40, 3-35, 3-30, 3-25, 3-20, 3-15, 3-10, 3-5, 4-99, 4-92, 4-90, 4-85, 4-80, 4-75, 4-70, 4-65, 4-60, 4-55, 4-50, 4-45, 4-40, 4-35, 4-30, 4-25, 4-20, 4-15, 4-10, 4-5, 5-99, 5-95, 5-90, 5-85, 5-80, 5-75, 5-70, 5-65, 5-60, 5-55, 5-50, 5-45, 5-40, 5-35, 5-30, 5-25, 5-20, 5-15, 5-10, 6-99, 6-92, 6-90, 6-85, 6-80, 6-75, 6-70, 6-65, 6-60, 6-55, 6-50, 6-45, 6-40, 6-35, 6-30, 6-25, 6-20, 6-15, 6-10, 10-99, 10-95, 10-90, 10-85, 10-80, 10-75, 10-70, 10-65, 10-60, 10-55, 10-50, 10-45, 10-40, 10-35, 10-30, 10-25, 10-20, 10-15, 15-99, 15-95, 15-90, 15-85, 15-80, 15-75, 15-70, 15-65, 15-60, 15-55, 15-50, 15-45, 15-40, 15-35, 15-30, 15-25, 15-20, 20-99, 20-95, 20-90, 20-85, 20-80, 20-75, 20-70, 20-65, 20-60, 20-55, 20-50, 20-45, 20-40, 20-35, 20-30, 20-25, 25-99, 25-95, 25-90, 25-85, 25-80, 25-75, 25-70, 25-65, 25-60, 25-55, 25-50, 25-45, 25-40, 25-35, 25-30, 30-99, 30-95, 30-90, 30-85, 30-80, 30-75, 30-70, 30-65, 30-60, 30-55, 30-50, 30-45, 30-40 or 30-35 wt %, at least about 1, 2, 3, 4, 5, 6, 7, 8, 9, 10, 15, 20, 25, 30, 35, 40, 45, 50, 55, 60, 65, 70, 75, 80, 85, 90, 95 or 99 wt %, or no more than about 1, 5, 10, 15, 20, 25, 30, 35, 40, 45, 50, 55, 60, 65, 70, 75, 80, 85, 90, 95 or 99 wt %, based on the total weight of the outer layer.

According to the preparation method, the solid fat may be have a melting point of about 25-90, 25-85, 25-80, 25-75, 25-70, 25-65, 25-60, 25-55, 25-50, 25-45, 25-40, 25-35, 25-30, 30-90, 30-85, 30-80, 30-75, 30-70, 30-65, 30-60, 30-55, 30-50, 30-45, 30-40, 30-35, 35-90, 35-85, 35-80, 35-75, 35-70, 35-65, 35-60, 35-55, 35-50, 35-45, 35-40, 40-90, 40-85, 40-80, 40-75, 40-70, 40-65, 40-60, 40-55, 40-50, 40-45, 45-90, 45-85, 40-80, 40-75, 40-70, 40-65, 40-60, 40-55, 40-50, 40-45, 45-90, 45-85, 45-80, 45-75, 45-70, 45-65, 45-60, 45-55, 45-50, 50-90, 50-85, 50-80, 50-75, 50-70, 50-65, 50-60, 50-55, 55-90, 55-85, 55-80, 55-75, 55-70, 55-65, 55-60, 60-90, 60-85, 60-80, 60-75, 60-70, 60-65, 65-90, 65-85, 65-80, 65-75, 65-70, 70-90, 70-85, 70-80, 70-75, 75-90, 75-85, 75-80, 80-90, 80-85 or 85-90° C. The solid fat may be from plants, algae, fungi, yeast, bacteria, or animals. The solid fat may be a hydrocarbon, which may be a natural product or synthetic. The solid fat may be an animal solid fat selected from the group consisting of beef tallow (mp 35-38° C.), mutton tallow (mp 40-45° C.), butter, cholesterol esters, stearine (mp 49-75° C.) and stearic acid (mp 71° C.). The solid fat may be a vegetable solid fat selected from the group consisting of hydrogenated oil, coconut oil, coconut butter, cocoa butter, cocoa butter equivalents, palm oil kernel, and phytosterol esters. The solid fat may be a natural wax selected from the group consisting of carnauba wax (mp 78-81° C.), candelilla wax (mp 68° C.), beeswax (mp 60-63° C.), spermaceti-sperm oil (mp 42-49° C.), Japan wax, jojoba oil and hardened jojoba oil (mp 30-40° C.). Examples of hydrocarbons (unsaponifiable) include paraffin wax (mp 35-36 C), montan wax (mp 76-84° C.), Ceresine wax (mp 60-85° C.), solid polyethylene glycols (Carbowax, PEGs>1000), and metallic stearates (e.g., aluminum stearates, magnesium stearates, sodium stearates, zinc stearate, calcium stearate). The solid fat is selected from the group consisting of animal fats, vegetable fats, natural waxes, hydrocarbon waxes, metallic stearates, solid polyethylene glycols (PEGs>1000) and a combination thereof. The solid fat may be edible. Examples of edible solid fats include coconut oil, coconut butter, cocoa butter, cocoa butter equivalents, palm oil kernel, stearic acid and stearic acid salts, and phytosterol esters and a combination thereof.

In one embodiment, according to the preparation method, the double-layer coated microparticle comprises a core, an inner layer and an outer layer. The core is coated with the inner layer and the outer layer. The inner layer is between the core and the outer layer. The core comprises the bioactive substance at 4-99 wt % of the weight of the core, the inner layer comprises a hydrophobic antioxidant at 1-80 wt % of the weight of the inner layer, and the outer layer comprises a solid fat at 20-95 wt % of the weight of outer layer.

According to the preparation method, the outer layer may be in a solid form in the coated microparticles, which may be incorporated into a food product containing the coated microparticles, when being stored. The final melting point of the outer layer may be adjusted by mixing two or more solid fats having different melting points. The outer layer in the coated microparticle may be converted from a liquid form into solid form when the temperature is reduced, for example, below room temperature (e.g., 0-25° C.), through cooling. The outer layer in the coated microparticle may remain in a solid form when the coated microparticles are stored or incorporated into a food product. The coated microparticle may be stored at a temperature below the melting point of the solid fat. The coated microparticle may be stored at a temperature of about 0-90, 0-85, 0-80, 0-75, 0-70, 0-65, 0-60, 0-55, 0-50, 0-45, 0-40, 0-0, 25-30, 30-90, 30-85, 30-80, 30-75, 30-70, 30-65, 30-60, 30-55, 30-50, 30-45, 30-40, 30-35, 35-90, 35-85, 35-80, 35-75, 35-70, 35-65, 35-60, 35-55, 35-50, 35-45, 35-40, 40-90, 40-85, 40-80, 40-75, 40-70, 40-65, 40-60, 40-55, 40-50, 40-45, 45-90, 45-85, 40-80, 40-75, 40-70, 40-65, 40-60, 40-55, 40-50, 40-45, 45-90, 45-85, 45-80, 45-75, 45-70, 45-65, 45-60, 45-55, 45-50, 50-90, 50-85, 50-80, 50-75, 50-70, 50-65, 50-60, 50-55, 55-90, 55-85, 55-80, 55-75, 55-70, 55-65, 55-60, 60-90, 60-85, 60-80, 60-75, 60-70, 60-65, 65-90, 65-85, 65-80, 65-75, 65-70, 70-90, 70-85, 70-80, 70-75, 75-90, 75-85, 75-80, 80-90, 80-85 or 85-90° C., below about 20, 25, 30, 35, 40, 45, 50, 55, 60, 65, 70, 75, 80, 85 or 90° C., or above 1, 5, 10, 15, 20, 25, 30, 35, 40, 45, 50, 55, 60, 65 or 70° C.

According to the preparation method, the double-layer coated microparticle, the inner layer may be in contact with the core, and the outer layer may be in contact with the inner layer. The double-layer coated microparticle may be dry. The double-layer coated microparticle may have a water activity (Aw) no more than 0.5, 0.3, 0.2 or 0.1.

The first contacting step may be carried out at a temperature of about 25-90, 25-85, 25-80, 25-75, 25-70, 25-65, 25-60, 25-55, 25-50, 25-45, 25-40, 25-35, 25-30, 30-90, 30-85, 30-80, 30-75, 30-70, 30-65, 30-60, 30-55, 30-50, 30-45, 30-40, 30-35, 35-90, 35-85, 35-80, 35-75, 35-70, 35-65, 35-60, 35-55, 35-50, 35-45, 35-40, 40-90, 40-85, 40-80, 40-75, 40-70, 40-65, 40-60, 40-55, 40-50, 40-45, 45-90, 45-85, 40-80, 40-75, 40-70, 40-65, 40-60, 40-55, 40-50, 40-45, 45-90, 45-85, 45-80, 45-75, 45-70, 45-65, 45-60, 45-55, 45-50, 50-90, 50-85, 50-80, 50-75, 50-70, 50-65, 50-60, 50-55, 55-90, 55-85, 55-80, 55-75, 55-70, 55-65, 55-60, 60-90, 60-85, 60-80, 60-75, 60-70, 60-65, 65-90, 65-85, 65-80, 65-75, 65-70, 70-90, 70-85, 70-80, 70-75, 75-90, 75-85, 75-80, 80-90, 80-85 or 85-90° C.

The second contacting step may be carried out at a temperature above the melting point of the solid fat. The second contacting step may be carried out at a temperature of about 25-90, 25-85, 25-80, 25-75, 25-70, 25-65, 25-60, 25-55, 25-50, 25-45, 25-40, 25-35, 25-30, 30-90, 30-85, 30-80, 30-75, 30-70, 30-65, 30-60, 30-55, 30-50, 30-45, 30-40, 30-35, 35-90, 35-85, 35-80, 35-75, 35-70, 35-65, 35-60, 35-55, 35-50, 35-45, 35-40, 40-90, 40-85, 40-80, 40-75, 40-70, 40-65, 40-60, 40-55, 40-50, 40-45, 45-90, 45-85, 40-80, 40-75, 40-70, 40-65, 40-60, 40-55, 40-50, 40-45, 45-90, 45-85, 45-80, 45-75, 45-70, 45-65, 45-60, 45-55, 45-50, 50-90, 50-85, 50-80, 50-75, 50-70, 50-65, 50-60, 50-55, 55-90, 55-85, 55-80, 55-75, 55-70, 55-65, 55-60, 60-90, 60-85, 60-80, 60-75, 60-70, 60-65, 65-90, 65-85, 65-80, 65-75, 65-70, 70-90, 70-85, 70-80, 70-75, 75-90, 75-85, 75-80, 80-90, 80-85 or 85-90° C.

The preparation method may further comprise storing the double-layer coated microparticles. The storage may be carried out at a temperature below the melting temperature of the solid fat. The storage may be carried out under predetermined storage conditions for a predetermined period of time. The predetermined storage conditions may include a predetermined temperature and a predetermined relative humidity (RH). The term “relative humidity (RH)” as used herein refers to the amount of water vapor in the air, often at a given temperature. Relative humidity is usually less than that required to saturate the air, and is often expressed in percentage of saturation humidity. The predetermined temperature may be at least about 0, 5, 10, 15, 20, 25, 37, 40, 45, 50 or 55° C. The predetermined relative humidity (RH) may be at least about 10%, 20%, 30%, 33%, 35%, 40%, 50%, 60%, 70% or 80%. The predetermined conditions may be accelerated storage conditions. For example, the predetermined conditions may include about 40° C. and about 33% RH, or about 45° C. and about 33% RH. The predetermined period of time may be at least about 1, 2, 3 or 4 weeks, or 1, 2, 3, 4, 5, 6, 12, 18, 24 or 36 months, preferably at least about 1, 2 or 3 months, more preferably at least about 1 or 3 months. A specified time period may include a shorter or longer time period that is within 10% of the specified time period. The term “3 months” as used herein refers to a time period of about 84-90 days. The term “2 months” as used herein refers to a time period of about 56-60 days. The term “1 month” as used herein refers to a time period of about 28-30 days.

According to the preparation method, after storage, at least about 10, 15, 20, 25, 30, 35, 40, 45, 50, 55, 60, 65, 70, 75, 80, 85, 90, 95 or 99% of the double-layer coated microparticle remain intact.

According to the preparation method, after storage, the double-layer coated microparticles may retain about 1-99, 1-92, 1-90, 1-85, 1-80, 1-75, 1-70, 1-65, 1-60, 1-55, 1-50, 1-45, 1-40, 1-35, 1-30, 1-25, 1-20, 1-15, 1-10, 1-5, 5-99, 5-95, 5-90, 5-85, 5-80, 5-75, 5-70, 5-65, 5-60, 5-55, 5-50, 5-45, 5-40, 5-35, 5-30, 5-25, 5-20, 5-15, 5-10, 10-99, 10-95, 10-90, 10-85, 10-80, 10-75, 10-70, 10-65, 10-60, 10-55, 10-50, 10-45, 10-40, 10-35, 10-30, 10-25, 10-20, 10-15, 15-99, 15-95, 15-90, 15-85, 15-80, 15-75, 15-70, 15-65, 15-60, 15-55, 15-50, 15-45, 15-40, 15-35, 15-30, 15-25, 15-20, 20-99, 20-95, 20-90, 20-85, 20-80, 20-75, 20-70, 20-65, 20-60, 20-55, 20-50, 20-45, 20-40, 20-35, 20-30, 20-25, 25-99, 25-95, 25-90, 25-85, 25-80, 25-75, 25-70, 25-65, 25-60, 25-55, 25-50, 25-45, 25-40, 25-35, 25-30, 30-99, 30-95, 30-90, 30-85, 30-80, 30-75, 30-70, 30-65, 30-60, 30-55, 30-50, 30-45, 30-40 or 30-35 wt %, at least about 1, 2, 3, 4, 5, 6, 7, 8, 9, 10, 15, 20, 25, 30, 35, 40, 45, 50, 55, 60, 65, 70, 75, 80, 85, 90, 95 or 99 wt %, or no more than about 1, 5, 10, 15, 20, 25, 30, 35, 40, 45, 50, 55, 60, 65, 70, 75, 80, 85, 90, 95 or 99 wt % of the total weight of the double-layer coated microparticles.

According to the preparation method, after storage, the double-layer coated microparticles may retain about 1-99, 1-92, 1-90, 1-85, 1-80, 1-75, 1-70, 1-65, 1-60, 1-55, 1-50, 1-45, 1-40, 1-35, 1-30, 1-25, 1-20, 1-15, 1-10, 1-5, 5-99, 5-95, 5-90, 5-85, 5-80, 5-75, 5-70, 5-65, 5-60, 5-55, 5-50, 5-45, 5-40, 5-35, 5-30, 5-25, 5-20, 5-15, 5-10, 10-99, 10-95, 10-90, 10-85, 10-80, 10-75, 10-70, 10-65, 10-60, 10-55, 10-50, 10-45, 10-40, 10-35, 10-30, 10-25, 10-20, 10-15, 15-99, 15-95, 15-90, 15-85, 15-80, 15-75, 15-70, 15-65, 15-60, 15-55, 15-50, 15-45, 15-40, 15-35, 15-30, 15-25, 15-20, 20-99, 20-95, 20-90, 20-85, 20-80, 20-75, 20-70, 20-65, 20-60, 20-55, 20-50, 20-45, 20-40, 20-35, 20-30, 20-25, 25-99, 25-95, 25-90, 25-85, 25-80, 25-75, 25-70, 25-65, 25-60, 25-55, 25-50, 25-45, 25-40, 25-35, 25-30, 30-99, 30-95, 30-90, 30-85, 30-80, 30-75, 30-70, 30-65, 30-60, 30-55, 30-50, 30-45, 30-40 or 30-35 wt %, at least about 1, 2, 3, 4, 5, 6, 7, 8, 9, 10, 15, 20, 25, 30, 35, 40, 45, 50, 55, 60, 65, 70, 75, 80, 85, 90, 95 or 99 wt %, or no more than about 1, 5, 10, 15, 20, 25, 30, 35, 40, 45, 50, 55, 60, 65, 70, 75, 80, 85, 90, 95 or 99 wt % of the total weight of the bioactive substance.

According to the preparation method, after storage, the double-layer coated microparticles may retain about 1-99, 1-92, 1-90, 1-85, 1-80, 1-75, 1-70, 1-65, 1-60, 1-55, 1-50, 1-45, 1-40, 1-35, 1-30, 1-25, 1-20, 1-15, 1-10, 1-5, 5-99, 5-95, 5-90, 5-85, 5-80, 5-75, 5-70, 5-65, 5-60, 5-55, 5-50, 5-45, 5-40, 5-35, 5-30, 5-25, 5-20, 5-15, 5-10, 10-99, 10-95, 10-90, 10-85, 10-80, 10-75, 10-70, 10-65, 10-60, 10-55, 10-50, 10-45, 10-40, 10-35, 10-30, 10-25, 10-20, 10-15, 15-99, 15-95, 15-90, 15-85, 15-80, 15-75, 15-70, 15-65, 15-60, 15-55, 15-50, 15-45, 15-40, 15-35, 15-30, 15-25, 15-20, 20-99, 20-95, 20-90, 20-85, 20-80, 20-75, 20-70, 20-65, 20-60, 20-55, 20-50, 20-45, 20-40, 20-35, 20-30, 20-25, 25-99, 25-95, 25-90, 25-85, 25-80, 25-75, 25-70, 25-65, 25-60, 25-55, 25-50, 25-45, 25-40, 25-35, 25-30, 30-99, 30-95, 30-90, 30-85, 30-80, 30-75, 30-70, 30-65, 30-60, 30-55, 30-50, 30-45, 30-40 or 30-35%, at least about 1, 2, 3, 4, 5, 6, 7, 8, 9, 10, 15, 20, 25, 30, 35, 40, 45, 50, 55, 60, 65, 70, 75, 80, 85, 90, 95 or 99%, or no more than about 1, 5, 10, 15, 20, 25, 30, 35, 40, 45, 50, 55, 60, 65, 70, 75, 80, 85, 90, 95 or 99% of the activity of the bioactive substance.

The preparation method may further comprise agglomerating, extruding or spray drying a mixture of the bioactive substance and an agglomerating agent to form the core.

The preparation method may further comprise making a product with the double-layer coated microparticle. The product may be selected from the group consisting of pharmaceutical products, nutraceutical products, food products, feed products, and additive products for agriculture. The food products may be bakery products.

For each preparation method, a double-layer coated microparticle prepared according to the method is provided.

### Example 1. Preparation of Coated Vitamin A Microparticles

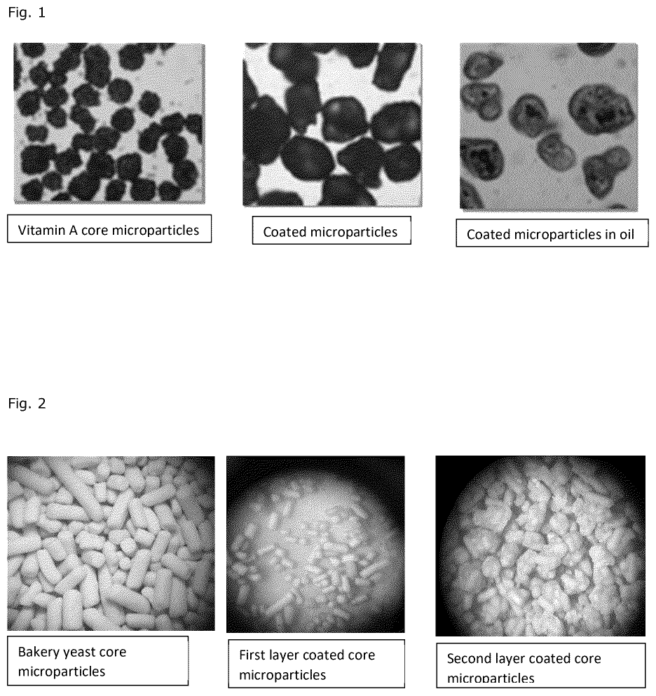

A solution was prepared by dissolving 10 g sucrose and 1 g gum acacia in 100 g warm water (40-60° C.). To this solution, was added 50 mg of sodium citrate, and the resulting agglomeration solution was cooled to room temperature. In a modified fluid bed dryer/granulator/coater system (Fluid Air model 2, 2 liter max working capacity), equipped with air blower, variable air velocity and variable heat control, 1000 g Vitamin A palmitate in the form of fine powder (BASF, Florham Park, NJ, particle size range from 0.5 micron to 10 micron) was agglomerated by top spraying the solution in fine mist for about 5 minutes with a two-fluid nozzle at an air pressure of 20 psi and a liquid rate of about 4 ml/min. The resulting vitamin A core microparticles (FIG. 1, left panel) were air dried in the fluidized bed dryer to a residual moisture level of less than 3% in the fluidized bed dryer using a drying temperature in the range from 50° C. to 60° C. The core microparticles were then coated with an inner layer followed by an outer layer to form coated microparticles (FIG. 1, middle panel). First, the core microparticles were top sprayed with an inner layer solution to form an inner layer over the core microparticles until the mass of the resulting microparticle increased by 20 wt % of the mass of the core microparticles. The inner layer solution was composed of 35 wt % of Rosemary extract (OxyLess, Naturex, Mamaroneck, NY), 40 wt % soy lecithin (Archer Daniels Midland Company, Decatur, IL), and 25 wt % canola oil (obtained from a local market). Then, an outer layer solution was then applied onto the microparticles coated by the inner layer to form an outer layer until the mass of the resulting coated microparticles increased by 100 wt % of the mass of the core microparticles. The outer layer solution was composed of 50 wt % stearic acid (Acros organics, NJ), 30 wt % palm kernel oil (Columbus Vegetable oils, Des Plaines, IL) and 20 wt % magnesium stearate (Sigma). The temperature of the outer layer solution was maintained at 70° C. throughout the coating process. The temperature of the resulting coated microparticles was maintained at about 40° C. Coated microparticles in a particle size range of 500-1500 μm were collected (FIG. 1, middle panel).

The collected coated microparticles were subject to a disintegration test in oil solution. Coated microparticles were mixed in canola oil for about 2 hours at room temperature and the mixture observed under microscope and evaluated for particle integrity. Over 80% of the observed coated microparticles remained intact in oil and their original particle size range of 500-1,500 μm was maintained.

The microparticles may also be produced in a batch process. During the process of coating with the inner or outer layer solution in the fluidized bed drier, the microparticles may be harvested periodically from the fluidized bed drier. Harvested microparticles having a mass or particle size less than a desirable mass or particle size would be returned to the fluidized bed drier to continue with the coating process.

### Example 2. Preparation of Coated Instant Bakery Yeast Microparticles

Using a coating pan such as commonly used in the pharmaceutical industry to coat tablets, 600 g of an instant dry bakery yeast (IDY, obtained from local market) was loaded as core microparticles (FIG. 2, left panel) in a pan. The pan was set to rotate at 60 to 75 RPM. A two-fluid nozzle was connected to a hot air supply, which was regulated between 15 to 25 psi. An inner layer solution containing 68 wt % Vitamin E acetate, 30 wt % canola oil, and 2 wt % Lecithin (Table 1) was prepared and maintained at about 40-45° C. The inner layer solution was supplied to the liquid side of the nozzle at a rate of about 6 ml/min. Warm air (40° C.) was blown into the pan to help facilitate a uniform coating of the IDY core microparticles (FIG. 2, middle panel). The inner layer solution was sprayed and deposited onto the core microparticles to be about 10 wt % of the core microparticles. The coating solution was then switched to an outer layer solution. The outer layer solution contained 50 wt % stearic acid, 33 wt % palm kernel oil, and 17 wt % magnesium stearate (Table 1). The outer layer solution was applied to constitute an additional 90 wt % of the core microparticles (FIG. 2, right panel). The temperature of the outer layer solution was maintained at 70° C. throughout the spraying process and the microparticle temperature was maintained at about 40-45° C. Coated IDY microparticles having a particle size range between 500 and 1500 μm were collected.

The coated microparticles may also be produced in a batch process. During the process of coating with the inner or outer layer solution in the coating pan, the microparticles may be harvested periodically. Harvested microparticles having a mass or particle size less than a desirable mass or particle size would be returned to the coating pan to continue with the coating process

TABLE 1
Coating Layers

| | wt % | |
|---|---|---|
| Layer | (weight) | Ingredients (wt %) |
| Inner | 10 wt % | 30 wt % canola oil, 68 wt % Vitamin E |
| | (60 g) | acetate and 2 wt % de-oiled soy lecithin. |
| Outer | 90 wt % | 33 wt % Palm kernel oil, 50 wt % stearic |
| | (540 g) | acid and 17 wt % magnesium stearate. |

Dispensability Assessments. The coated IDY microparticles were subjected to a dispensability assessment, in which the coated microparticles were mixed in deionized water (diH₂O) to determine duration required for complete degradation of the coated microparticles. 0.5 g of the coated microparticles were added in a culture tube (13×100 mm) containing 4.5 g diH₂O. The contents in the tube were mixed in a vortex mixer (speed setting of 7, Fisher Scientific, Model No. 945404) for 30 second intervals until a homogenous dispersion was observed. The corresponding IDY core microparticles were used as a negative control. The coated microparticles required about 3×30 sec mixing intervals to reach a homogeneous dispersion while the corresponding uncoated core microparticles required about 2×30 sec mixing intervals. Thus, the double-layer coating of IDY lowered IDY solubility and stabilized IDY.

### Example 3. Assessments of Coated Instant Dry Yeast Microparticles

Three coated microparticle samples each containing 25 g bakery yeast were prepared. Instant dry yeast (IDY) obtained from a local market were used as core microparticles (control sample) to be coated. Sample 1 was prepared by coating the IDY core microparticles with a single layer solution containing 100 wt % solid fats, PEG-4000. Sample 2 was prepared by coating the IDY core microparticles with a single layer solution, containing 2 wt % Vitamin E acetate 6 wt %, de-oiled soy lecithin, 20 wt % PEG-4000, 25 Palm kernel oil, 27 wt % stearic acid and 20 wt % magnesium stearate. Sample 3 was prepared by coating the IDY core microparticles with an inner layer solution followed by an outer layer solution, and the inner layer solution contained 11 wt % canola oil, 22 wt % Vitamin E acetate and 67 wt % de-oiled soy lecithin while the outer layer solution contained 89 wt % PEG-4000 and 11 wt % magnesium stearate. Table 2 provides the coating compositions for samples 1-3.

TABLE 2
Coating Compositions for Samples 1, 2 and 3

| | | wt % | |
|---|---|---|---|
| Layer | Sample | (weight) | Ingredients (wt %) |
| Single | 1 | 100 wt % | 100 wt % PEG-4000 |
| Single | 2 | 91 wt % | 2 wt % Vitamin E acetate 6 wt %, |
| | | (22.75 g) | de-oiled soy lecithin, 20 wt % PEG-4000 |
| | | | 25 wt % Palm kernel oil, 27 wt % stearic |
| | | | acid and 20 wt % magnesium stearate. |
| Inner | 3 | 9 wt % | 11 wt % canola oil, 22 wt % Vitamin E |
| | | (2.25 g) | acetate and 67 wt % de-oiled soy lecithin |
| Outer | 3 | 91 wt % | 89 wt % PEG-4000 and 11 wt % |
| | | (22.75 g) | magnesium stearate |

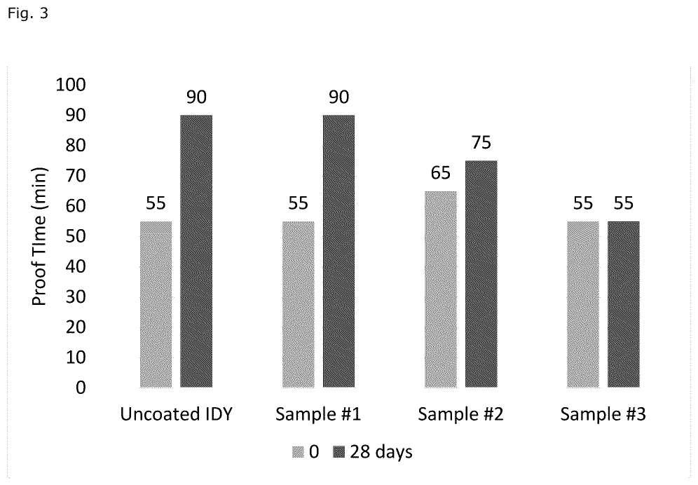

Samples 1, 2 and 3 and the control sample were stored in a desiccator at 25° C. and 53% relative humidity (RH) for 28 days before being assessed for IDY's activity as evidenced by the gassing power in flour dough based on proof time (FIG. 3), i.e., time to increase dough volume by four times. As shown in FIG. 3, the IDY microparticles with the double-layer coating in Sample 3 according to the present invention showed an initial gassing power of 50-55 min, which was the same as the control, and retained the initial gassing power after being stored at 25° C. and 53% RH for 28 days. Thus, as compared with the uncoated IDY (control) and the single-layer coated IDY (Samples 1 and 2), the double-layer coating (Sample 3) unexpectedly protected the IDY and preserved the IDY activity.

### Example 4. Assessments of Coated Instant Dry Yeast Microparticles in an Acidic Medium

Unprotected instant dry yeast (IDY) rapidly loses its activity, as evidenced by its gassing power, in an acidic dough, such as sourdough. The protection effect of the double-layer coating according to the present invention on the yeast gassing power as evidenced by proof time was evaluated. The IDY core microparticles were coated with an inner layer solution followed by an outer layer solution. The inner layer solution contained 29 wt % Vitamin E acetate, 30 wt % canola oil, and 41 wt % lecithin. The outer layer contained 45 wt % palm kernel, 40 wt % stearic acid and 15 wt % magnesium stearate. In the resulting double-layer coated microparticles, the inner and outer layers constituted about 10 wt % and 90 wt % of the mass of the core microparticles, respectively.

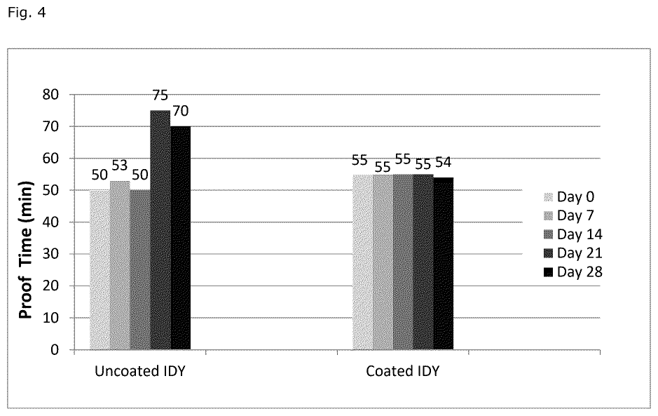

For gassing power stability assessment in an acidic medium, three grams (3 g) coated IDY microparticles and 1.5 g uncoated IDY microparticles, i.e., IDY core microparticles as control, were dispersed in a 10-ml solution containing 5 wt % gelatin at a pH of 3.7 and a temperature of about 45° C. The resulting dispersions were allowed to solidify at room temperature and then stored at about 4° C. for 0, 7, 14, 21 or 30 days before being tested for gassing power based on its proof time. As shown in FIG. 4, the proof time of the coated IDY microparticles remain about the same after storage up to 30 days while the proof time of the uncoated IDY microparticles remained about the same after storage up to 14 days, but increased by about 40-50% after storage for 21 or 28 days. Thus, as compared with the uncoated IDY, the double-layer coating unexpectedly protected the IDY and preserved the IDY activity after storage for at least 28 days.

### Example 5. Coated Savinase Microparticles in Baked Muffin Dough

Coated and uncoated savinase (Sigma-Aldrich Co., St. Louis, Missouri) will be prepared to make a muffin dough. To increase particle size of savinase, an agglomeration solution composed of 10 wt % maltodextrin, 2 wt % gum acacia and 0.5 wt % ascorbic acid (all from Sigma-Aldrich Co., St. Louis, Missouri) will be sprayed onto savinase to form savinase core microparticles. The core microparticles will then be coated in a fluidized bed drier or coating pan with an inner layer solution followed by an outer layer solution as described in Examples 1 or 2, respectively, to make coated savinase microparticles. The inner layer solution will be composed of 10 wt % Rosemary extract (OxyLess, Naturex, Mamaroneck, NY), 60 wt % soy oil carrier, and 30 wt % lecithin (Archer Daniels Midland Company, Decatur, IL). The outer layer solution will be composed of 45 wt % palm kernel, 40 wt % stearic acid and 15 wt % magnesium stearate. In the resulting double-layer coated microparticles, the inner and outer layers will constitute about 10 wt % and 90 wt % of the mass of the core microparticles, respectively.

The coated and uncoated savinase microparticles will be each mixed with muffin flour (General Mills Inc., Minneapolis, MN) to make a muffin dough having savinase at a final concentration of 20,000 IU/muffin. The muffin dough will be baked at 180° C. for 20 minutes. It is expected that no or very little remaining savinase activity will be detected in the baked muffin prepared with the uncoated savinase microparticles, and some savinase activity, for example, about 20-25%, will be retained in the baked muffin prepared with the coated savinase microparticles. Thus, as compared with the uncoated savinase, the double-layer coating will protect the savinase in the baked muffin and retention of, for example, about 20-25% of, the savinase activity after baking.

### Example 6. Coated Vitamin A Microparticles in Baked Cookies

Coated and uncoated vitamin A microparticles will be prepared as described in Example 1 and then mixed into Betty Crocker cookie flour (General Mills Inc., Minneapolis, MN) to make cookie doughs with a final concentration of vitamin A at 850 IU/g.

The cookie doughs will be baked at 200° C. for 20 minutes. It is expected that the concentration of vitamin A in the baked cookies prepared with uncoated vitamin A microparticles will drop significantly after baking while the concentration of vitamin A in the baked cookies prepared with the coated vitamin A microparticles will remain about the same after baking. Thus, as compared with the uncoated vitamin A, the double-layer coating will protect vitamin A in baked cookies.

### Example 7. Coated Agricultural Microbe Microparticles after Dry Storage

Coated and uncoated microparticles of agricultural microbe Trichoderma atroviride (obtained from a commercial source) will be prepared. An agglomeration solution composed of 10 wt % maltodextrin, 2 wt % gum acacia and 0.5 wt % ascorbic acid (all from Sigma-Aldrich Co., St. Louis, Missouri) will be sprayed onto Trichoderma atroviride to form Trichoderma atroviride core microparticles. The core microparticles will then be coated in a fluidized bed drier or coating pan with an inner layer solution followed by an outer layer solution as described in Examples 1 or 2, respectively, to make coated Trichoderma atroviride microparticles. The inner layer solution will be composed of 10 wt % Rosemary extract (OxyLess, Naturex, Mamaroneck, NY) and 90 wt % soy oil carrier. The outer layer solution will be composed of 45 wt % palm kernel, 50 wt % stearic acid and 5 wt % magnesium stearate. In the resulting double-layer coated microparticles, the inner and outer layers will constitute about 10 wt % and 90 wt % of the mass of the core microparticles, respectively.

The coated and uncoated Trichoderma atroviride microparticles will each be mixed with maltodextrin with an initial viability of 1×10⁹ colony-forming unit per gram (CFU/g). The resulting mixtures will be stored in a desiccator at 33% RH and 30° C. before the viability of the remaining Trichoderma atroviride will be measured as CFU/g every 30 days for a period of 6 months. It is expected that the uncoated Trichoderma atroviride will lose, for example, about 4 logs CFU/g, after storage for 3 months, while the coated Trichoderma atroviride will lose, for example, only about one (1) log CFU/g, after storage for 6 months. Thus, as compared with the uncoated Trichoderma atroviride, the double-layer coating will provide more protection of Trichoderma atroviride after dry storage.

The term “about” as used herein when referring to a measurable value such as an amount, a percentage, and the like, is meant to encompass variations of ±20% or ±10%, more preferably ±5%, even more preferably ±1%, and still more preferably ±0.1% from the specified value, as such variations are appropriate.

All documents, books, manuals, papers, patents, published patent applications, guides, abstracts, and other references cited herein are incorporated by reference in their entirety. Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

1. A double-layer coated microparticle for protecting a bioactive substance, comprising a core, an inner layer and an outer layer, wherein the core is coated with the inner layer and the outer layer, the inner layer is between the core and the outer layer, the core comprises the bioactive substance, the inner layer comprises a hydrophobic antioxidant, and the outer layer comprises a solid fat.

2. The double-layer coated microparticle of claim 1, wherein the bioactive substance is selected from the group consisting of probiotic bacteria, yeast, fungi, viruses, vaccines, nutraceuticals, pharmaceuticals, agro-biologicals, agro-chemicals, vitamins, enzymes, and food additives.

3. The double-layer coated microparticle of claim 1, wherein the double-layer coated microparticle comprises the inner layer at 1-25 wt % of the double-layer coated microparticle.

4. The double-layer coated microparticle of claim 1, wherein the hydrophobic antioxidant is selected from the group consisting of tocopherols, ascorbyl palmitate, tetrahexyldecyl ascorbate, vitamin A and derivatives thereof, carotenoids, quercetin and derivatives thereof, phenolic compounds, phyto-nutrients, herbs extracts and a combination thereof.

5. The double-layer coated microparticle of claim 1, wherein the inner layer further comprises an emulsifier.

6. The double-layer coated microparticle of claim 1, wherein the double-layer coated microparticle comprises the outer layer at 20-95 wt % of the double-layer coated microparticle.

7. The double-layer coated microparticle of claim 1, wherein the solid fat is selected from the group consisting of animal fats, vegetable fats, natural waxes, hydrocarbon waxes, metallic stearates, solid polyethylene glycols (PEGs>1000) and a combination thereof.

8. The double-layer coated microparticle of claim 1, wherein the double-layer coated microparticle has a particle size of 500-10,000 μm.

9. The double-layer coated microparticle of claim 1, wherein the double-layer coated microparticle is stored at a temperature below the melting temperature of the solid fat.

10. A method for preparing a double-layer coated microparticle, comprising:
(a) contacting a core with an inner layer solution to form an inner layer on the core, wherein the core comprises a bioactive substance, and the inner layer comprises a hydrophobic antioxidant, whereby an inner-layer coated microparticle is obtained; and
(b) contacting the inner-layer coated microparticle from step (a) with an outer layer solution to form an outer layer on the inner-layer coated microparticle, wherein the outer layer comprises a solid fat, whereby a double-layer coated microparticle is prepared.

11. The method of claim 10, wherein step (a) is carried out at a temperature of 40-60° C.

12. The method of claim 10, wherein step (b) is carried out at a temperature above the melting temperature of the solid fat.

13. The method of claim 10, further comprising:
(c) storing the double-layer coated microparticle at a temperature below the melting temperature of the solid fat.

14. The method of claim 10, wherein the bioactive substance is selected from the group consisting of probiotic bacteria, yeast, fungi, viruses, vaccines, nutraceuticals, pharmaceuticals, agro-biological, agro-chemicals, vitamins, enzymes, and food additives.

15. The method of claim 10, further comprising agglomerating, extruding or spray drying a mixture of the bioactive substance and an agglomerating agent, whereby the core is formed and has a particle size of 50-5,000 μm.

16. The method of claim 10, wherein the double-layer coated microparticle has a particle size of 500-10,000 μm.

17. The method of claim 10, wherein the double-layer coated microparticle comprises the inner layer at 1-25 wt % of the coated microparticle.

18. The method of claim 10, wherein the hydrophobic antioxidant is selected from the group consisting of tocopherols, ascorbyl palmitate, tetrahexyldecyl ascorbate, vitamin A and derivatives thereof, carotenoids, quercetin and derivatives thereof, phenolic compounds, phyto-nutrients, herbs extracts and a combination thereof.

19. The method of claim 10, wherein the inner layer further comprises an emulsifier.

20-22. (canceled)

23. A double-layer coated microparticle prepared according to the method of claim 10.
